(19) United States
(12) Patent Application Publication
(10) Pub. No.: US 2009/0258035 A1
(43) Pub. Date: Oct. 15, 2009

(54) AVIAN VIRUS VACCINES AND USES THEREOF

(75) Inventors: Qingzhong Yu, Athens, GA (US); Carlos Estevez, Athens, GA (US); Daniel J. King, Athens, GA (US); David L. Suarez, Athens, GA (US); Patti J. Miller, Loganville, GA (US)

Correspondence Address: USDA-ARS-OFFICE OF TECHNOLOGY TRANSFER;PATENT ADVISORS OFFICE, WESTERN REGIONAL RESEARCH CENTER, 800 BUCHANAN ST, ALBANY, CA, 94710, US

(73) Assignee: The United States of America, as represented by the Secretary of Agriculture, Washington, DC (US)

(21) Appl. No.: 12/408,541

(22) Filed: Mar. 20, 2009

Related U.S. Application Data
(60) Provisional application No. 61/038,720, filed on Mar. 21, 2008.

(51) Int. Cl. A61K 39/17; A61P 37/04; A61P 31/12

(52) U.S. Cl. 424/214.1

(57) ABSTRACT

The present invention relates to poultry vaccines for prevention of Newcastle disease.

### REFERENCE TO RELATED APPLICATIONS

This application claims priority to U.S. Provisional Patent Application Ser. No. 61/038,720 filed Mar. 21, 2008, which is incorporated herein by reference in its entirety.

### FIELD OF THE INVENTION

The invention relates to poultry vaccines for protection from Newcastle disease.

### BACKGROUND OF THE INVENTION

Newcastle Disease Virus (NDV) is a diverse and deadly avian pathogen. NDV is the causative agent of Newcastle disease. Poultry infected with a virulent NDV typically develop respiratory and/or nervous symptoms that can be fatal (see e.g., D. J. Alexander (2003) Newcastle disease, other avian paramyxoviruses, and pneumovirus infections. In Saif, J. M., Barnes, H. J., Glisson, J. R., Fadly, A. M., McDougald, L. R., Swayne, D. E. (ed.), Diseases of Poultry. Iowa State University Press, Ames, pp. 63-87).

Newcastle disease may present with symptomology including, but are not limited to respiratory signs (e.g., gasping, coughing), nervous signs (e.g., depression, lack of appetite, drooping wings, paralysis), swelling of the eyes and neck, diarrhea, misshapen, rough- or thin-shelled eggs and reduced egg production. However, Newcastle disease may strike asymptomatically as well. Indeed, in such cases, non-vaccinated birds may be found dead without ever having shown signs of illness. In both symptomatic and asymptomatic Newcastle disease, postmortem examination may reveal characteristic lesions in the brain and/or gastrointestinal tract.

The disease can spread rapidly and can exact a heavy toll, therefore Newcastle disease is a clear and present danger to the commercial poultry industry. Indeed, during the 20th Century, the poultry industry has developed into an international industry dependent on intensive trade between countries. Thus, since Newcastle disease occurs on six of the seven continents, and is enzootic in many countries, the possibility of an outbreak of Newcastle disease is a constant threat.

The morbidity and mortality associated with Newcastle disease can be enormous. Newcastle disease viruses are classified into three pathotypic groups which describe their virulence in poultry: lentogenic (low virulence), mesogenic (moderate virulence), and velogenic (high virulence). The most severe form of Newcastle disease•(velogenic) can result in morbidity rates near 100 percent, and mortality rates as high as 90 percent in susceptible chickens. The most recent U.S. outbreak, which occurred in 2002-2003 in California, Nevada, Arizona and Texas, illustrates the devastation and financial cost that can result from an outbreak of Newcastle disease. In that outbreak, more than 3.4 million birds were depopulated and the cost for controlling the outbreak in California alone was more than 200 million dollars (see e.g., Kapczynski, D. R. (2005) Vaccine 23(26):3424-3433).

Vaccines for control of Newcastle disease have been used for over fifty years. The available vaccines do tend to reduce mortality and the severity of symptoms in infected birds. Unfortunately however, known vaccines do not prevent infection of susceptible birds, nor do they prevent or reduce shedding of the virus by already infected birds. Thus, the known vaccines do not prevent transmission of Newcastle disease virus.

Since available vaccines do not prevent transmission, Newcastle disease and its inevitable spread is still a feared event in the poultry industry. Therefore, what is needed in the art is a vaccine that will prevent transmission and/or reduce transmission rates and rates of infection of susceptible birds by Newcastle disease virus. Such a vaccine would prevent or reduce shedding of the virus by already infected birds and would prevent infection an/or reduce infection rates of susceptible birds.

Fortunately, as will be clear from the following disclosure, the present invention provides for these and other needs.

### SUMMARY OF THE INVENTION

In one aspect, the present invention provides a Newcastle Disease vaccine that comprises a recombinant attenuated virulence Newcastle disease virus; wherein the recombinant attenuated virulence Newcastle disease virus comprises at least one substituted gene that is homologus to a corresponding gene of a virulent challenge strain of Newcastle disease virus, and wherein the at least one substituted gene is a member selected from the group consisting of a hemagglutinin neuraminidase (HN) gene and a fusion (F) gene or a combination thereof.

In one exemplary embodiment, the Newcastle Disease vaccine is a live vaccine.

In another exemplary embodiment, the recombinant attenuated virulence Newcastle disease virus is a member selected from the group consisting of lentogenic Newcastle disease viruses and mesogenic Newcastle disease viruses.

In one exemplary embodiment, the recombinant attenuated virulence Newcastle disease virus is a lentogenic Newcastle disease virus that is a member selected from the group consisting of: Ulster/196, B1/1947, and LaSota/1946.

In another exemplary embodiment, the recombinant low virulence Newcastle disease virus is a mesogenic Newcastle disease virus. In another exemplary embodiment, the mesogenic Newcastle disease virus is recombinant Anhinga strain. In another exemplary embodiment, the recombinant Anhinga strain comprises a substituted HN gene. In another exemplary embodiment, the recombinant Anhinga strain further comprises a substituted F gene.

In a second aspect, the invention provides a method for stimulating immunity to Newcastle disease in poultry, the method comprises vaccinating poultry with a vaccine comprising a recombinant attenuated virulence Newcastle disease virus; wherein the recombinant attenuated virulence Newcastle disease virus comprises at least one substituted gene homologus to a virulent challenge strain of Newcastle disease virus, and wherein the at least one substituted gene homologus to the virulent challenge strain of Newcastle disease virus is a member selected from the group consisting of a hemagglutinin neuraminidase (HN) gene and a fusion (F) gene or a combination thereof.

In one exemplary embodiment, the vaccine is a live vaccine.

In one exemplary embodiment, the vaccinating is achieved through mass vaccination. In another exemplary embodiment, the mass vaccination is achieved by providing the vaccine in drinking water. In one exemplary embodiment, the mass vaccination is achieved by contacting the poultry with an aerosol spray comprising the vaccine.

In one exemplary embodiment, the vaccinating is achieved through subcutaneous injection. In one exemplary embodiment, the vaccine is inactivated. In another exemplary embodiment, the vaccine is live.

Other aspects, features, objects and advantages of the invention will be apparent from the detailed description which follows.

### BRIEF DESCRIPTION OF THE DRAWINGS

FIG. 1 Experiment I—Comparison of virus titers from oropharyngeal swabs for all vaccine groups collected on days (a) two and (b) four post-challenge with CA02 vNDV. Challenge occurred 21 days post-vaccination. * Indicates significant difference from Control-OE. ** Indicates significant difference between rA-OE and CA02-OE. P≦0.05

FIG. 2 Experiment I—ELISA titers from pre-challenge serum taken twenty-one days after vaccination from groups vaccinated with (a) inactivated oil-emulsion (OE) and (b) live vaccines. * Indicates significance from the sham-vaccinated controls. ** Indicates significance between vaccine groups. The 396 designates the cut off for a positive NDV antibody titer. P≦0.05

FIG. 3. Experiment I—Comparison of virus titers from oropharyngeal swabs for all vaccine groups collected on days (a) two and (b) four post-challenge with CA02 vNDV. Challenge occurred 21 days post-vaccination. * Indicates significant difference from Controls. ** Indicates significant difference between live B1 and live rA-CAHN. P≦0.05

FIG. 4 Experiment II—Virus isolation at four days post-vaccination from live vaccine groups in oropharyngeal swabs. * Indicates significance compared to sham-vaccinated controls. Vaccine groups contain both eventual vaccine challenge groups for each vaccine. P≦0.05

FIG. 5 Experiment II—Comparison of virus titers from oropharyngeal swabs for all vaccine groups collected on days (a) two and (b) four post-challenge with CA02 vNDV (CA) or TXGB (TX) vNDV. Challenge occurred at 21 days post-vaccination. * Indicates significant difference from sham-vaccinated controls. P≦0.05

FIG. 6 Experiment II—Comparison of virus titers from cloacal swabs for all vaccine groups collected on days (a) two and (b) four post-challenge with CA02 vNDV (CA) or TXGB (TX) vNDV. Challenge occurred at 21 days post-vaccination. * Indicates significant difference from sham-vaccinated controls. P≦0.05

FIG. 7 Experiment II—ELISA titers from pre-challenge serum taken twenty-one days after vaccination from groups vaccinated with live vaccines. * Indicates significance from the sham-vaccinated controls. The 396 designates the cut off for a positive NDV antibody titer. Vaccine groups contain both eventual vaccine challenge groups for each vaccine. P≦0.05

FIG. 8 Experiment III—Virus isolation from (a) oropharyngeal and (b) cloacal swabs from live vaccine groups at four days post-vaccination. * Indicates significance compared to sham-vaccinated controls. Vaccine groups contain both eventual vaccine challenge groups for each vaccine. P≦0.05.

FIG. 9 Experiment III—Comparison of virus titers from oropharyngeal swabs for all vaccine groups collected on days (a) two and (b) four post-challenge with CA02 vNDV (CA) or TXGB (TX) vNDV. Challenge occurred at 21 days post-vaccination. * Indicates significant difference from sham-vaccinated controls. ** Indicates significance between LaSota-TX and rA-TX and between rA-TX and rA-CAHN-CA. P≦0.05

FIG. 10 Experiment III—Comparison of virus titers from cloacal swabs for all vaccine groups collected on days (a) two and (b) four post-challenge with CA02 vNDV (CA) or TXGB (TX) vNDV. Challenge occurred at 21 days post-vaccination. * Indicates significant difference from sham-vaccinated controls. P≦0.05.

FIG. 11 Experiment III—ELISA titers from pre-challenge serum taken twenty-one days post-vaccination from groups vaccinated with live vaccines. * Indicates significance from the sham-vaccinated controls. The 396 designates the cut off for a positive NDV antibody titer. Vaccine groups contain both eventual vaccine challenge groups for each vaccine. P≦0.05.

FIG. 12 Experiment IV—Comparison of virus titers from oropharyngeal swabs for all vaccine groups collected on days (a) two and (b) four post-challenge with CA02 vNDV (CA) or TXGB (TX) vNDV. Challenge occurred at 21 days post-vaccination. * Indicates significant difference from sham-vaccinated controls. ** Indicates significance between LaSota-TX and LaSota-CA. *** Indicates significance between rA-CAFHN-TX and rA-CAFHN-CA. P≦0.05.

FIG. 13 Experiment IV—Comparison of virus titers from cloacal swabs for all vaccine groups collected on days (a) two and (b) four post-challenge with CA02 vNDV (CA) or TXGB (TX) vNDV. Challenge occurred at 21 days post-vaccination. * Indicates significant difference from sham-vaccinated controls. P≦0.05.

FIG. 14 Experiment IV—ELISA titers from pre-challenge serum taken twenty-one days after vaccination from groups vaccinated with live vaccines. * Indicates significance from the sham vaccinated controls and ** indicates significance of rA-CAFHN from LaSota. The 396 designates the cut off for a positive NDV antibody titer. Vaccine groups contain both eventual vaccine challenge groups for each vaccine. P≦0.05.

### DETAILED DESCRIPTION OF THE INVENTION

### Definitions

The term “Newcastle disease virus” or “NDV” as used herein, refers to avian Paramyxovirus type -1 virus which is a member of the genus Avulavirus in the Paramyxoviridae family. Newcastle disease virus is the causative agent of Newcastle disease. As is known in the art, strains of Newcastle disease viruses are all antigenically of the same serotype, but can be separated into genotypes based on genome differences. Genetic analysis of fully sequenced NDV strains reveals the existence of at least two distinct classes (Class I and Class II) and at least eight genotypes of NDV (see e.g., Czeglédi, A. et al. (2006) Virus Research 120 (1-2):36-48, which is incorporated herein by reference). Since the classes and genotypes are evolutionarily related, Newcastle disease viruses within a given lineage are “homologous” Newcastle disease viruses. Similarly, corresponding genes derived from “homologous” Newcastle disease viruses are “homologous”. For example, a hemaglutinin neuraminidase (HN) gene from a particular viral lineage is “homologous” with the HN gene of all other Newcastle disease viruses in that lineage.

The term “challenge strain” as used herein, is used broadly to refer to any infectious Newcastle disease virus that, by virtue of its ability to infect an organism and cause disease (of any severity) has the ability to test (i.e. challenge) the immune system of an organism (e.g., of a poultry species, e.g., a chicken) to respond to the Newcastle disease virus and fight off disease (e.g., fight off by preventing disease, minimizing or reducing disease symptoms over what could otherwise be expected and/or fight off by reducing, minimizing, or eliminating shed of challenge virus from infected individuals). The term “challenge strain” as used herein refers both to a Newcastle disease virus that is intentionally contacted with poultry e.g., under experimental, laboratory conditions, as well as to a Newcastle disease virus which is contacted with poultry in a fortuitous or unintentional manner outside the laboratory e.g., in Nature, at a breeding facility, etc.

Thus the expression “homologus to the challenge strain” as used herein, refers to a Newcastle disease virus or Newcastle disease virus gene, or fragment thereof, that is from the same lineage as the challenge strain and is therefore evolutionarily related to the challenge strain.

The term “virulent” as used herein is used broadly to refer to a Newcastle disease virus that is capable of causing disease and/or death by disease. The term “virulent” as used herein refers to the ability to cause disease without regard to the extent and/or severity of disease and/or disease symptoms. Thus, a “virulent challenge strain” is an infectious Newcastle disease virus that is capable of causing disease at least in un-vaccinated birds. The term “virulence” as used herein refers to the ability of a Newcastle disease virus to produce disease. The virulence of a Newcastle disease virus is a measure of the severity of the disease it is capable of causing. For example, as is known in the art, Newcastle disease virus occurs as three different pathotypes which reflect their virulence in poultry: lentogenic (low virulence), mesogenic (moderate virulence), and velogenic (high virulence). Virulence of Newcastle disease viruses can be determined using any one or more standard pathotyping assays known in the art (see e.g., deLeeuw et al. (2005) J. Gen. Virol. 86:1759-1769; N. Wakamatsu, et al. (2006)Virology 353:333-343; B. P. Peeters, et al. (2000) Arch. Virol. 145:1829-1845 and/or A. Romer-Oberdorfer, et al. (2003) J. Gen. Virol. 84:3121-3129).

Thus, the term “low virulence Newcastle disease virus” as used herein, refers to any Newcastle disease virus, either naturally occurring, or recombinant, which has “low virulence” as determined by any standard pathotyping assay known in the art.

The term “attenuated virulence Newcastle disease virus” as used herein refers to any Newcastle disease virus whose level of virulence is less than that of the high virulence (velogenic) pathotype as measured in a standard pathotyping assay known in the art. Attenuation may be naturally occurring, may be selected for in vitro or in vivo, or may be induced by recombinant methods e.g., site directed mutagenesis.

The term “poultry” as used herein, refers to domestic fowl, e.g., chickens, turkeys, guinea fowl, ducks, geese, quails, pigeons, game birds e.g., pheasants, partridges ratites, etc, that are reared or kept in captivity for breeding, the production of meat or eggs, or for restocking supplies of game.

The term “prevent” or “prevention” as used herein, refers to any indicia of success in prevention, or amelioration of Newcastle disease, and/or the symptoms of Newcastle disease, including any objective or subjective parameter such as abatement, remission, diminishing of symptoms, and prevention or lessening of neurological and/or gastrointestinal symptoms and/or prevention or lessening of neurological and/or gastrointestinal damage or injury. For example, the terms “prevent” or “prevention” as used herein, refer to the prevention of neurological lesions and/or damage, or reduction in the number or severity of lesions or damage; the prevention of gastrointestinal lesions and/or damage; elimination or reduction of viral shedding as compared to unvaccinated birds; reduction in expected deaths or in the death rate of birds infected with a virulent challenge strain. In an exemplary embodiment, success in the prevention of Newcastle disease is measured by comparing the symptomology, morbidity and/or mortality and/or viral shedding in vaccinated poultry to the symptomology, morbidity and/or mortality and/or viral shedding in un-vaccinated poultry, and observing that the severity or occurrence of symptoms and/or the morbidity and/or mortality and/or viral shedding of vaccinated poultry is lessened, diminished and therefore reduced by comparison to un-vaccinated poultry. In another exemplary embodiment, success in the prevention of Newcastle disease is measured by comparing the symptomology, morbidity and/or mortality and/or viral shedding in poultry vaccinated with a standard Newcastle disease vaccine (e.g., B1, LaSota, etc) to the symptomology, morbidity and/or mortality in poultry vaccinated with a recombinant Newcastle disease virus that comprises at least one substituted gene that is homologous to a corresponding gene in the virulent challenge strain and observing that the severity or occurrence of symptoms and/or the morbidity and/or mortality of poultry vaccinated with the recombinant Newcastle disease virus that comprises at least one substituted gene that is homologous to a corresponding gene in the virulent challenge strain is lessened, diminished and therefore reduced by comparison to poultry vaccinated with a standard Newcastle disease vaccine. The prevention, treatment, reduction or amelioration of symptoms can be based on objective or subjective parameters; including the results of physical examination, biopsy or microscopic examination of a tissue sample, or any other appropriate means known in the art.

The term “reduce” as used herein refers to any indicia of success in the diminishment in size, amount, extent, severity or number of neurological and/or gastrointestinal lesions or in the damage associated with lesions and/or diminishment in amount, extent, severity or number of any of the symptoms of Newcastle disease including but not limited to of neurological and/or gastrointestinal lesions. The term “reduce” as used herein also refers to any indicia of success in the diminishment in of morbidity, mortality, viral shed and/or spread of Newcastle disease in vaccinated populations; including, but not limited to diminishment of spread (e.g., rate or extent of spread) from an un-vaccinated infected population to a vaccinated population; and including e.g., diminishment of spread from a vaccinated infected population to an un-vaccinated population.

The term “biological sample” as used herein refers to any sample obtained from a living or dead organism. Examples of biological samples include isolated organs or body parts e.g., isolated spleen, trachea, thymus; mucosa, swabs e.g., cloacal swabs oropharyngeal swabs.

The terms “isolated,” “purified,” or “biologically pure” as used herein, refer to material that is substantially or essentially free from components that normally accompany it as found in its native state. In one exemplary embodiment, an isolated Newcastle disease virus is a virus that is cultured, or synthesized and then cultured in vitro (see e.g., Estevez, C. et al. (2007) Virus Research 129:182-190, which is incorporated herein by reference). In another exemplary embodiment, purity is determined using analytical chemistry techniques such as polyacrylamide gel electrophoresis or high performance liquid chromatography. A nucleic acid that is the predominant species present in a preparation is substantially purified. In one exemplary embodiment, an isolated nucleic acid is a nucleic acid that is separated from open reading frames and/or other nucleic acid sequences that flank the isolated nucleic in its native state. In another exemplary embodiment, the term “purified” denotes that a nucleic acid or protein gives rise to essentially one band in an electrophoretic gel. Typically, isolated nucleic acids or proteins have a level of purity expressed as a range. The lower end of the range of purity for the component is about 60%, about 70% or about 80% and the upper end of the range of purity is about 70%, about 80%, about 90% or more than about 90%.

The term “biologically pure culture” as used herein, refers to a continuous in vitro culture of Newcastle Disease virus which is substantially free of other organisms. A culture is substantially free of other organisms if standard harvesting procedures (as described herein) result in a preparation which comprises at least about 90%, at least about 95%, preferably at least about 99% or more of the organism, e.g., at least about 90% Newcastle disease virus.

The term “nucleic acid” as used herein, refers to a polymer of ribonucleotides or deoxyribonucleotides. Typically, “nucleic acid” polymers occur in either single- or double-stranded form, but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (see e.g., Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “polypeptide,” “peptide” and “protein” are used interchangeably herein to refer to a polymer of amino acid residues. The terms apply to naturally occurring amino acid polymers and non-naturally occurring amino acid polymers as well as amino acid polymers in which one or more amino acid residues is an artificial chemical mimetic of a corresponding naturally occurring amino acid.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids are referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art (see, e.g., Creighton, Proteins (1984)). Such conservatively modified variants are in addition to and do not exclude polymorphic variants, interspecies homologs, and alleles.

The following eight groups illustrate some exemplary amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K);
5) Isoleucine (1), Leucine (L), Methionine (M), Valine (V);
6) Phenylalanine (F), Tyrosine (Y), Tryptophan (W);
7) Serine (S), Threonine (T); and
8) Cysteine (C), Methionine (M)

Macromolecular structures such as polypeptide structures are described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The term “label” as used herein, refers to a composition detectable by spectroscopic, photochemical, biochemical, immunochemical, or chemical means. Exemplary labels include ³²P, fluorescent dyes, electron-dense reagents, enzymes (e.g., as commonly used in an ELISA), biotin, digoxigenin, or haptens and proteins for which antisera or monoclonal antibodies are available.

As used herein a “nucleic acid probe or oligonucleotide” refers to a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (e.g., 7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. In one exemplary embodiment, probes are directly labeled as with isotopes, chromophores, lumiphores, chromogens etc. In other exemplary embodiments probes are indirectly labeled e.g., with biotin to which a streptavidin complex may later bind. By assaying for the presence or absence of the probe, one can detect the presence or absence of the select sequence or subsequence.

Thus, the term “labeled nucleic acid probe or oligonucleotide” as used herein refers to a probe that is bound, either covalently, through e.g., a linker or a chemical bond, or noncovalently, through ionic, van der Waals, electrostatic, or hydrogen bonds to a label such that the presence of the probe may be detected by detecting the presence of the label bound to the probe.

The term “primer” as used herein, refers to short nucleic acids, typically DNA oligonucleotides of at least about 15 nucleotides in length. In an exemplary embodiment, primers are annealed to a complementary target DNA strand by nucleic acid hybridization to form a hybrid between the primer and the target DNA strand. Annealed primers are then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

PCR primer pairs are typically derived from a known sequence, for example, by using computer programs intended for that purpose such as Primer (Version 0.5© 1991, Whitehead Institute for Biomedical Research, Cambridge, Mass.). One of skill in the art will appreciate that the specificity of a particular probe or primer increases with its length. Thus, for example, a primer comprising 20 consecutive nucleotides of Newcastle disease virus (NDV) sequence will anneal to a related target sequence with a higher specificity than a corresponding primer of only 15 nucleotides. Thus, in an exemplary embodiment, greater specificity of a nucleic acid primer or probe, is attained with probes and primers selected to comprise 20, 25, 30, 35, 40, 50 or more consecutive nucleotides of a selected sequence.

Nucleic acid probes and primers are readily prepared based on the nucleic acid sequences disclosed herein. Methods for preparing and using probes and primers and for labeling and guidance in the choice of labels appropriate for various purposes are discussed, e.g., in Sambrook et al., Molecular Cloning, A Laboratory Manual 2nd ed. 1989, Cold Spring Harbor Laboratory; and Current Protocols in Molecular Biology, Ausubel et al., eds., 1994, John Wiley & Sons). The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, over expressed, under expressed or not expressed at all.

Isolated NDV sequences can be isolated from any source and/or can be synthetically made, by methods known on the art (see e.g., U.S. Pat. No. 5,942,609 and Estevez, C. et al. (2007), supra) as long as they are substantially identical to NDV sequences as disclosed herein and/or as known in the art. Methods for determining nucleotide sequence identity and “substantial identity” are described herein below. However, in general, nucleic acid sequences are considered to be substantially identical when the nucleic acid sequences or their complements hybridize to each other under stringent hybridization conditions, as described below.

The term “capable of hybridizing under stringent hybridization conditions” as used herein, refers to annealing a first nucleic acid to a second nucleic acid under stringent hybridization conditions (defined below). In an exemplary embodiment, the first nucleic acid is a test sample, and the second nucleic acid is the sense or antisense strand of NDV. Hybridization of the first and second nucleic acids is conducted under standard stringent conditions, e.g., high temperature and/or low salt content, which tend to disfavor hybridization of dissimilar nucleotide sequences.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “transformation” as used herein encompasses any and all techniques by which a nucleic acid molecule might be introduced into a cell, including but not limited to, transfection with viral vectors, transformation with plasmid vectors, and introduction of naked DNA by electroporation, lipofection, etc.

The following terms are used to describe the sequence relationships between two or more nucleic acids or polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length NDV sequence or gene sequence given in a sequence listing, or may comprise a complete NDV sequence or gene sequence.

The terms “identical” or percent “identity,” in the context of two or more nucleic acids or polypeptide sequences, refer to two or more sequences or subsequences that are the same or have a specified percentage of amino acid residues or nucleotides that are the same (e.g., 85% identity, 90% identity, 99%, or 100% identity), when compared and aligned for maximum correspondence over a comparison window, or designated region as measured using a sequence comparison algorithm or by manual alignment and visual inspection.

The phrase “substantially identical”, in the context of two nucleic acids or polypeptides, refers to two or more sequences or subsequences that have at least about 85%, identity, at least about 86%, 87%, 88%, 89%, 90%, 91%, 92%, 93%, 94%, 95%, 96%, 97%, 98%, 99%, or 100% nucleotide or amino acid residue identity, when compared and aligned for maximum correspondence, as measured using a sequence comparison algorithm or by visual inspection. In an exemplary embodiment, the substantial identity exists over a region of the sequences that is at least about 50 residues in length. In another exemplary embodiment, the substantial identity exists over a region of the sequences that is at least about 100 residues in length. In still another exemplary embodiment, the substantial identity exists over a region of the sequences that is at least about 150 residues or more, in length. In one exemplary embodiment, the sequences are substantially identical over the entire length of nucleic acid or protein sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An exemplary algorithm for sequence comparison is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12:387-395 (1984).

Another example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a nucleic acid molecule only to a particular target nucleotide sequence under stringent hybridization conditions when that particular nucleotide sequence is present in a complex mixture (e.g., total cellular or library DNA or RNA). For example in general, two nucleic acid sequences are said to be “substantially identical” when the two molecules or their complements selectively or specifically hybridize to each other under stringent conditions. Although a nucleic acid molecule that “specifically hybridizes to” a particular target nucleotide sequence may also bind to unrelated sequences, typically at least about 90%, at least about 91%, at least about 92%, at least about 93%, at least about 94%, at least about 95%, or more of the hybridization complexes formed by the nucleic acid molecule are with the target nucleotide sequence.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology-Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. However, other high stringency hybridization conditions known in the art can be used.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

The term “antibody” as used herein, refers to an immunoglobulin molecule able to bind to a specific epitope on an antigen. Antibodies can be a polyclonal mixture or monoclonal. Antibodies can be intact immunoglobulins derived from natural sources or from recombinant sources and can be immunoreactive portions of intact immunoglobulins. Antibodies may exist in a variety of forms including, for example, Fv, F_{ab}, and F(ab)₂, as well as in single chains. Single-chain antibodies, in which genes for a heavy chain and a light chain are combined into a single coding sequence, may also be used.

The term “antigen” as used herein, refers to a molecule that is recognized and bound by an antibody, e.g., a peptide, carbohydrates, organic molecules, glycolipids and/or glycoproteins. The part of the antigen that is the target of antibody binding is referred to as an “antigenic determinant” and a small functional group that corresponds to a single “antigenic determinant” is referred to as a hapten.

The phrase “specifically immunoreactive with”, when referring to a protein or peptide, refers to a binding reaction between the protein and an antibody which is determinative of the presence of the protein in the presence of a heterogeneous population of proteins and other compounds. Thus, under designated immunoassay conditions, the specified antibodies bind to a particular protein and do not bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein and are described in detail below.

The phrase “pharmaceutically acceptable” as used herein, means a material that within the scope of sound veterinary or medical judgment, is not biologically or otherwise undesirable, i.e., the material can be administered to an animal along with NDV vaccine components and adjuvants without undue toxicity, irritation, allergic response, and the like, commensurate with a reasonable benefit/risk ratio, thus, without causing any undesirable biological effects or interacting in a deleterious manner with any of the other components of a pharmaceutical composition.

### I. Introduction:

As noted above, Newcastle disease is a scourge of the poultry industry. Thus, vaccines that are effective for prevention of Newcastle disease are highly desired. Fortunately, it has now been discovered that a Newcastle disease vaccine that is effective for the prevention of Newcastle disease is prepared from a recombinant Newcastle disease virus that comprises a homologous substitution of the hemagglutinin neuraminidase (HN) or the HN and fusion (F) gene, wherein the homologous substitution(s) are homologous to the corresponding genes of a virulent challenge strain.

### II. Newcastle Disease Virus

Newcastle Disease Virus (NDV), also known as avian Paromyxovirus type-1, is a member of the genus Avulavirus in the family Paramyxoviridae. NDV is an avian-paramyxovirus serotype-1. There are eight other serotypes of avian-paramyxovirus, designated avian-paramyxovirus type-2 to -9. These are distinguished on the basis of their antigenic relatedness in hemagglutination-inhibition tests and serum neutralization tests (see e.g., Alexander, D. J., (1993) Paramyxovirus infections. In Virus infections of birds. McFerran, J. B. and McNulty, M. S. (eds), pp 321-340, Elsevier Science Publishers).

The genome of NDV is a single-stranded, non-segmented RNA virus of negative polarity, complementary to the messenger RNA's which code for the virus proteins. The RNA genome is about 15,200 nucleotides in size with variation apparent between different lineages (Czeglédi, A. (2006) supra) The genome codes for six known gene products (listed from the 3′ end to the 5′ end of the genomic RNA): nucleocapsid protein (NP), phosphoprotein (P), matrix protein (M), fusion protein (F), hemagglutinin-neuraminidase (HN), and large polymerase protein (L). RNA editing of the P protein produces two additional proteins V and W (see e.g., Chambers, P. et al., (1986) J. Gen. Virol. 67: 475-486).

The hemagglutinin (HN) and fusion (F) are transmembrane surface glycoproteins that function in binding and fusion of the virus to host cells to initiate an NDV infection. The HN and F induce specific cell-mediated immunity and neutralizing antibodies in vaccinated animals. Thus, antibodies to HN and F are the primary protective factors induced by NDV vaccines (see e.g., Seal, B. S. et al (2000) Dev. Comp. Immunol. 24 (2-3):257-268).

Typically, NDV enters the body of susceptible species, e.g., commercial domestic poultry e.g., chicken, turkey, pheasant, guinea fowl, duck, goose, pigeon; captive, semi-domestic and free-living birds e.g., migratory waterfowl; via the respiratory and the intestinal tract or via the eye. In the trachea, the virus is spread by ciliary action and by cell-to-cell spread. After initial multiplication at the introduction site, virus is carried during episodes of viremia to spleen, liver, kidney and lungs. Viruses of some strains reach vital organs like liver and kidney very rapidly so that the birds may die before disease symptoms are overt.

Replication of NDV is similar to that of other paramyxovirinae. The initial step is attachment of the virus to the host cell receptors, mediated by the HN protein. Fusion of the viral envelope with the host cell membrane is dependent on the action of both the HN and F proteins and results in the release of the RNP into the cytoplasm where virus replication takes place (see e.g., Lamb, R. A. and Kolakofsky, D. (1996) Paramyxoviridae: the viruses and their replication. In: Fundamental Virology (Fields et al., eds), Chapter 20, 577-604, Lipincott-Raven Publishers, Philadelphia.).

During the replication of NDV the precursor glycoprotein Fo is cleaved to F1 and F2 thereby producing infectious virus (see e.g., Rott, R. and Klenk, H. D. (1988) J. Gen. Virol. 50:135-147). This posttranslational cleavage is mediated by host cell proteases. If cleavage fails to take place, non-infectious virus particles are produced and viral replication cannot proceed. The Fo protein of virulent viruses can be cleaved by a wide range of proteases, but Fo proteins in viruses of low virulence are restricted in their sensitivity and these viruses can only grow in vivo in certain host cell types and in general cannot be grown in vitro. Thus, the F gene product is generally thought to be a major determinant of virulence, however a complete understanding of virulence mechanisms remains unknown (see e.g., Estevez, C. (2007) supra).

As is well known in the art, and as noted above, Newcastle disease viruses are classified into three pathotypes groups which describe their virulence in poultry: lentogenic (low virulence), mesogenic (moderate virulence), and velogenic (high virulence). These pathotypes are readily distinguished by persons of skill utilizing standard pathotypic assays (see e.g., (see e.g., deLeeuw et al. (2005) supra; N. Wakamatsu, et al. (2006) supra; B. P. Peeters, et al. (2000) supra; A. Romer-Oberdorfer, et al. (2003) supra and U.S. Pat. No. 7,332,169).

Typically, lentogenic viruses only replicate in areas with trypsin-like enzymes such as the respiratory and intestinal tract, whereas virulent viruses can replicate in a range of tissues and organs resulting in fatal systemic infection.

As is well known in the art, because ND viruses all belong to a single serotype, and thus, any NDV vaccine strain can protect from clinical disease and mortality from any NDV challenge virus (see e.g., Butterfield, W. K. et al. (1973) Avian Dis. 17(2):279-282). Therefore, a collection of standard low virulence viruses are used to prepare live vaccines against Newcastle disease. Some exemplary standard vaccine strains include e.g., strains B1, LaSota and Ulster.

Unfortunately however, the standard vaccine strains do not provide the same level of protection against each challenge strain. Indeed, over time, strains of virulent NDV have developed which are able to infect chickens vaccinated with standard vaccines (see e.g., Kapcznski D. R. (2005) Vaccine 23(26):3424-3433 and Spatlin, J. and Hanson, R. P. (1972) Proceedings of the 21^{st} Western poultry disease conference and 6^{th} poultry health symposium pgs. 66-69) thereby raising questions about the efficacy of the standard vaccines.

Furthermore, although NDV's are of the same serotype, antigenic and genetic diversity are recognized within the serotype (see e.g., Czeglédi, A. et al. (2006), supra; Alexander D. J. et al. (1997) Vet. Rec. 143(8):209-212; Aldous, E. W. et al. (2003) Avian Pathol. 32(3):239-256 and Ballagi-Pordany, A. et al. (1996) Arch Virol. 141(2):243-261). Indeed, recent genetic analysis has revealed at least eight genotypes of NDV which are evolutionarily related (see e.g., Czeglédi, A. et al. (2006) supra). Also, it has been shown increased effectiveness of vaccines to prevent shed of virus from vaccinated and subsequently infected birds, when the vaccine is prepared from a Newcastle disease virus that is related (i.e., homolgous) to the challenge strain Moreover, the degree of prevention correlates to the closeness of relationship. In particular, the more closely related the vaccine is to the challenge strain, the less virus is shed by the vaccinated birds after infection with the challenge strain (see e.g., Miller, P. J., et al. (2007) Vaccine 25(41):7238-7246, which is incorporated herein by reference).

Unfortunately, however high virulence NDV cannot be used as live vaccine, for obvious reasons. As is well known in the art, mass vaccination of poultry flocks typically requires live, low virulence Newcastle disease virus. Therefore what is needed in the art are effective Newcastle disease virus vaccines that have restricted or attenuated levels of virulence, such that they can be used as either live or inactivated vaccines.

### III. Isolating NDV and Constructing Recombinant Vaccines

A. General Recombinant DNA Methods

This invention utilizes routine techniques in the field of recombinant genetics. Basic texts disclosing the general methods of use in this invention include Sambrook et al., Molecular Cloning—A Laboratory Manual (2nd Ed.), Vol. 1-3, Cold Spring Harbor Laboratory, Cold Spring Harbor, N.Y., 1989; Kriegler, Gene Transfer and Expression: A Laboratory Manual (1990); and Current Protocols in Molecular Biology (Ausubel et al., eds., 1994) each of which is incorporated herein by reference). Unless otherwise noted, technical terms are used according to conventional usage. Definitions of common terms in molecular biology maybe found in e.g., Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

For nucleic acids, sizes are given in either kilobases (kb) or base pairs (bp). Estimates are typically derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized e.g., according to the solid phase phosphoramidite triester method first described by Beaucage & Caruthers, Tetrahedron Letts. 22:1859-1862 (1981), using an automated synthesizer, as described in Van Devanter et al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The sequence of the cloned genes and synthetic oligonucleotides can be verified after cloning using, e.g., the chain termination method for sequencing double-stranded templates of Wallace et al., Gene 16:21-26 (1981) or any other convenient method known in the art see e.g., L. M. Kim, et al. (2006) J Vet Diagn Invest 18(6):519-528 which is incorporated herein by reference.

B. Methods for the Isolation of Full Length NDV

Methods for the isolation and identification of avian pathogens, including NDV are known in the art. See e.g., Alexander, D. J. (1998) Newcastle Disease Virus and other Avian Paramyxoviruses. In Swayne, D. E., Glisson, J. R., Jackwood, M. W., Pearson, J. E., Reed, W. M. (ed.), A Laboratory Manual for the Isolation and Identification of Avian Pathogens. American Association of Avian Pathologists, Kennett Square, which is incorporated herein by reference).

Methods for the isolation of full length Newcastle disease virus genome, the construction of plasmids comprising full length NDV genome; construction of plasmids comprising select NDV genes and methods for rescue of infectious recombinant or non-recombinant NDV clones are known in the art. See e.g., Estevez, C. et al. (2007) supra; A. Czeglédi, et al. (2002) Epidemiol. Infect. 129:679-688; and U.S. Pat. No. 7,332,169.

As is readily appreciated by the skilled practitioner, any method known in the art can be used to produce a full length ND virus backbone, which can be modified by any convenient method or combination of methods known in the art and disclosed herein to produce the effective Newcastle disease vaccines disclosed herein.

C. Sequence Features of NDV

NDV is the only member of serotype I of avian paramyxoviruses (APMV-1). NDV possess single-stranded continuous RNA genome of negative polarity consisting of over 15,000 nucleotides. The genome of NDV contains six genes in the order of 3′-NP-P-M-F-HN-L-5′ that code for six major polypeptides (nucleoprotein, phosphoprotein, matrix, fusion, hemagglutinin-neuraminidase and large, respectively).

Numerous isolates of NDV have been sequenced, thus the sequence features of the NDV genome are known (see e.g. Czeglédi, A. et al. (2006) supra).

### IV. Immunization

A. Vaccine Administration

Vaccines against Newcastle disease are well known in the art. Typically vaccines are formulated for administration as either live vaccines or as inactivated vaccines, and can be prepared by any means known in the art (see e.g., U.S. Pat. No. 6,348,197; Stone (1978) Avian Dis 22: 666-74; Fwu-Long Mi et al. (1999) Biomaterials 20(17):1603-1612; etc).

In the U.S., and in many countries worldwide, ND prevention is focused on bio-security and the vaccination of poultry with both live and inactivated ND vaccines. As is known in the art vaccines are typically administered after maternal antibodies have waned which allows the induction of a good immunological response before the birds are likely to be exposed to a virulent strain of NDV. A person of skill in the art is aware that both live and inactivated vaccines have their advantages and disadvantages (see e.g., D. A. Senne, et al. (2004), Dev Biol (Base1) 119:165-170) and will be able to weigh the factors that concern them most and thereby choose the best formulation for their own purposes.

In an exemplary embodiment, a recombinant Newcastle disease virus vaccine is formulated as a live vaccine. Live vaccines have are able to be easily mass applied, are less expensive to produce and are able to induce mucosal immunity through IgA production (see e.g., Jayawardane, G. W. and Spradbrow, P. B. (1995). Vet Microbiol, 46, 37-41). In one exemplary embodiment, the live vaccine is administered by a mass application technique. A common mass application method is via drinking water. In other exemplary embodiments, the live vaccine is mass applied by contacting a population of poultry with a spray or aerosol application as is known in the art. Spray and/or aerosol application may be used for any size population, however, for large numbers of birds, it is known that spray and/or aerosol application allows for easy vaccination of large many birds in a short time. In some exemplary embodiments vaccine is delivered by combining it with food that is thrown to the poultry.

In other exemplary embodiments, a recombinant Newcastle disease virus vaccine is formulated as an inactivated vaccine. Inactivated vaccines are well known in the art. See e.g., Miller, P. J. et al. (2007) supra.

### V. Virus Assay

A. Serologic Assay

The presence of virus is detected by any convenient method known in the art. Newcastle disease virus, being a paramyxovirus, causes hemagglutination of chicken red blood cells. Thus, in one exemplary embodiment, viral presence is determined using a hemagglutination activity assay. Hemagglutination activity assay is known in the art (see e.g., U.S. Pat. No. 5,118,502 and N. Wakamatsu, et al. (2006) Vet Pathol 43(6):925-933).

In another exemplary embodiment, viral presence is determined using a hemagglutination-inhibition assay. Hemagglutination-inhibition assay is known in the art (see e.g., W. W. Marquardt, et al. (1985) Avian Diseases, Vol. 29, No. 1, pp. 71-79; and D. J. King (1996) Avian Dis 40(1):210-217.).

B. Neutralization Assay

By using a standard amount of virus, a standard amount of blood cells and serially diluting the antiserum, one can identify the minimum inhibitory concentration of the antiserum (the greatest dilution which inhibits hemagglutination). See e.g., Thayer S G, and Beard, C. W. 1998. Serological Procedures. In A Laboratory Manual for the Isolation and Identification of Avian Pathogens, ed. D E Swayne, pp. 255-66. Kennett Square: American Association of Avian Pathologists, which is incorporated herein by reference.

The following examples are offered to illustrate, but not to limit the invention.

### EXAMPLES

### Material and Methods for Examples 1-4

Eggs and Chickens.

The Southeast Poultry Research Laboratory (SEPRL) specific pathogen-free (SPF) white Leghorn flock was the source of the four week-old chickens used in all of the vaccination experiments. Embryonated chicken eggs (ECE) from SEPRL were utilized for virus isolation (VI), vaccine propagation, virus titrations, and as a source of normal allantoic fluid for preparing both the inactivated sham vaccine for the control birds and for diluting antigens after inactivation for the inactivated vaccines. Additional ECE from Sunrise Farm, Inc. (Catskill, N.Y.) were used for virus titrations. Chickens were separated into appropriate vaccination groups for each experiment. Number of birds per group differed for each of the experiments ranging from 6 to 20. The chickens were wing or leg banded and kept in negative pressure isolation units in BSL 3 Ag facilities and allowed to acclimate for 2 days prior to their being vaccinated. ELISA (IDEXX, Westbrook, Me.) and hemagglutination inhibition (HI) assays were completed on sera from hatchmates from each experimental group to confirm the absence of NDV antibodies. Birds were given food and water ad libitum throughout the experiment. The SEPRL Institutional Animal Care and Use Committee approved all animal experiments.

Viruses.

Working stocks of virus isolates used as vaccines were obtained from the SEPRL repository and propagated in 9-11 day old SPF ECE by chorioallantoic sac inoculation. Newcastle disease viruses representing different genotypes were selected to use as vaccines or challenge viruses: Ulster/196, B1/1947, LaSota/1946, TXGB/1948 and California/212676/2002 (CA02) (Alexander, (2003) Newcastle disease, other avian paramyxoviruses, and pneumovirus infections. In Diseases of Poultry, ed. J M Saif, Barnes, H. J., Glisson, J. R., Fadly, A. M., McDougald, L. R., Swayne, D. E., pp. 63-87. Ames: Iowa State University Press; Wakamatsu et al., (2006) Vet Pathol 43: 925-33) (see Table 1). Ulster, a class II genotype I virus, is used as a live vaccine in Northern Ireland and B1 and LaSota, both class II genotype II viruses, are used worldwide as live vaccines. TXGB is the virulent neurotropic challenge virus used to test efficacy of NDV vaccines in the U.S. and is a class II, genotype II virus and is the same genotype as B1 and LaSota. In these experiments TXGB is used as one of the challenge viruses. The CA02 viscerotropic virus, a class II, genotype V virus, is a virulent representative of the last outbreak of vNDV in the U.S. and was used as an inactivated vaccine and challenge virus. In addition, recombinant viruses created from the backbone of a genotype V NDV isolated from an Anhinga in 1993, which is in the same class and genotype as CA02 were used as both live and inactivated vaccines (King, D. J., and Seal, B. S. (1998) Avian Dis 42: 507-16). Some of these recombinants are chimeras that contain genes from the CA02 virus instead of their own genes, and their creation and characterization has been previously described (Estevez et al., 2007, supra). The strain of virus used for the HN and F genes is CA/212519/2002 has the same sequence as the CA02 virus in the HN and F genes, but was collected from a different chicken during the same outbreak. Because the sequence of the HN and F genes is the same they both will be referred to as CA02. An additional recombinant containing the HN gene of CA02 in the Anhinga backbone along with the fusion gene cleavage site of LaSota (rA-CAHN-LSCL) was evaluated as a vaccine in experiments II and III. Pools of infective allantoic fluid were clarified via centrifugation at 1000×g for 15 minutes. Infectivity titers of the pools were determined by titration in ECE prior to being stored at −70° C. for use as live vaccine virus and prior to being inactivated for use as antigen for inactivated vaccines and for HI assays. Hemagglutination-(HA) titers were determined before and after inactivation (data not shown) (Thayer, S. G. and Beard, C. W. (1998) supra). Allantoic fluid for each virus was inactivated with 0.1% beta-propiolactone (BPL) (Sigma, St. Louis, Mo.) (Spradbrow, P. B. and Samuel, J. L. (1991) Aust Vet J 68: 114-5) for 4 hours at room temperature and kept overnight at 4° C. for hydrolysis of the BPL. Complete virus inactivation was confirmed by failure to recover virus after embryo inoculation (Alexander, 1998). Prior to being stored at −70° C., the pH of the pools of virus antigen as allantoic fluid were adjusted to 7.0 by adding sterile sodium bicarbonate solution (Gibco, Invitrogen Corporation, Grand Island, N.Y.) (Budowsky, E. I., et al., (1993) Vaccine 11: 343-8). Inactivated antigen was used for the inactivated vaccines and for the HI assays.

Vaccine Generation.

Water-in-oil emulsion (OE) vaccines were prepared with virus antigen concentration the equivalent of 10^{8.3} ELD₅₀ (median embryo lethal dose) of virus prior to BPL inactivation. Recombinant Anhinga (rA), having a lower ELD₅₀ titer and HA titer, was concentrated by ultra-centrifugation at 120,000×g. The oil phase of the vaccine was made by adding 36 parts of Drakeol 6VR (Butler, Pa.), 3 parts of Span 80 (Sigma, St. Louis, Mo.) and 1 part of Tween 80 (Sigma, St. Louis, Mo.) for each vaccine to be made into a working solution. The oil phase was added to each of the virus antigens or normal allantoic fluid (the aqueous phase) to achieve a 4:1 ratio of oil to water as previously described (Stone, H. D. (1983) Avian Dis 27: 688-97). Vaccines were prepared by homogenization in a Waring blender (Fisher Scientific International Inc., Hampton, N.H.) (Stone, H. D., et al., (1978) V) three days prior to administration and kept at 4° C. prior to use. Infected allantoic fluid was clarified by centrifugation at 1000×g for 15 minutes and diluted in brain heart infusion (BHI) broth (BD Biosciences, Sparks, Md.) to an EID₅₀ of 10⁶/0.1 ml for formulation of the live virus vaccines. All sham vaccines given to the control birds in the live vaccine experiments contained sterile BHI.

Experimental Design.

Viruses used in each experiment are summarized in Table 1.

TABLE 1
Experimental design summaries

| | | |
|---|---|---|
| | Challenge(s) | # birds/ |
| Vaccines | (dose/bird) | group |

Experiment I

| | | | |
|---|---|---|---|
| Part I-Inactivated Oil Emulsion | CA02 | (10^{5.7}/0.1 ml) | 6 |
| A) control-OE | | | |
| B) rA-OE | | | |
| C) rA-CAHN-OE | | | |
| D) CA02-OE | | | |
| Part II-Live | | | |
| A) control-BHI | | | |
| B) B1 | | | |
| C) rA | | | |
| D) rA-CAHN | | | |

Experiment II

| | | | |
|---|---|---|---|
| Live | CA02 | (10^{5.9}/0.1 ml) | 6 |
| A) control-BHI | TXGB | (10^{6.1}/0.1 ml) | |
| B) LaSota | | | |
| C) rA | | | |
| D) rA-CAHN | | | |
| E) rA-CAHN-LSCL | | | |

Experiment III

| | | | |
|---|---|---|---|
| Live | CA02 | (10^{7.1}/0.1 ml) | 10 |
| | TXGB | (10^{6.3}/0.1 ml) | |
| A) control-BHI | | | |
| B) Ulster | | | |
| C) LaSota | | | |
| D) rA | | | |
| E) rA-CAHN | | | |
| F) rA-CAFHN | | | |
| G) rA-CAHN-LSCL | | | |

Experiment IV

| | | | |
|---|---|---|---|
| Live | CA02 | (10^{6.9}/0.1 ml) | 20 |
| | TXGB | (10^{6.5}/0.1 ml) | |
| A) LaSota | | | |
| B) rA-CAFHN | | | |

The first experiment (Experiment I) assessed both inactivated (part 1) and live (part 2) vaccines containing conventional viral strains and recombinant viruses to a challenge with vNDV, CA02. Experiment II evaluated only live vaccines including an additional recombinant. A second challenge virus, TXGB, that is genotypically different than CA02 was added to further test how homology between vaccine and challenge virus affected the amount of the virus shed post-challenge. In experiment III larger numbers of birds were used per group, a conventional class II, genotype I vaccine (Ulster) dissimilar to both of the challenge viruses was added, a new recombinant (rA-CAFHN) was also used as a vaccine and the challenge dose per bird was increased. The last experiment assessed the commonly used LaSota vaccine and the recombinant vaccine, rA-CAFHN, against two high dose challenges with TXGB and CA02 with bird groups of twenty. In the second and third experiments, extra birds were vaccinated for each of the vaccine groups and tissues were harvested four days post-vaccination (PV) for VI to recover the vaccine viruses. Spleen and blood was harvested for experiment II and tracheas and lungs were harvested in the third experiment. For all of the experiments, OP and CL were collected on days 0, 2, 4, and 9 into 1.5 ml of BHI broth with a final concentration of gentamicin (200 μg/ml), penicillin G (2000 units/ml), and amphotericin B (4 μg/ml). Birds were monitored daily for clinical signs and death through day 14 PC when they were sedated, bled and euthanized. Moribund chickens were euthanized with intravenous sodium pentobarbital at a dose of 100 mg/kg and counted as dead on the next day. Necropsies were completed on selected birds post-challenge to assess the presence of gross pathological lesions.

VI, EID₅₀, HA, HI, and ELISA

Virus isolation (VI) and hemagglutination (HA) assays to identify virus positive fluids were conducted as described (Miller et al., (2007) Vaccine 25(41): 7238-7246; Wakamatsu et al., 2006, supra). Tissues collected for VI were homogenized with BHI and VI antibiotics (described above) in a Whirlpak (NASCO, Modesto, Calif.) with a Stomacher 80 (Seward, West Sussex, UK) for two minutes to obtain a 10% weight: volume suspension. All VI positive samples were titrated in SPF ECE (Alexander DJ. (1998) Newcastle Disease Virus and other Avian Paramyxoviruses. In A Laboratory Manual for the Isolation and Identification of Avian Pathogens, ed. DE Swayne, Glisson, J. R., Jackwood, M. W., Pearson, J. E., Reed, W. M., pp. 156-63. Kennett Square: American Association of Avian Pathologists) and virus titers were calculated to determine the EID₅₀ using the Spearman-Kärber method (Karber, (1931) Arch. Exp. Pathol. Pharnak. 162: 480-3). Hemagglutination-inhibition (HI) assays (micro-beta) were completed on pre-challenge sera by testing all samples against their homologous and heterologous vaccine antigens (King, D. J. (1996) Avian Dis 40: 210-7). ELISA assays (IDEXX, Westbrook, Me.) were also completed on the pre-challenge serum according to the manufacturer's recommendations. Arithmetic mean titers with standard errors of HI antibodies were determined for each vaccination group.

Site Directed Mutagenesis.

Site directed mutagenesis was performed on clones of the already rescued recombinant virus, rA-CAHN to change the fusion cleavage site of the backbone of the Anhinga fusion gene to the less virulent cleavage site of the LaSota virus (Estevez et al., 2007, supra). Briefly, using PfuUltra® II Fusion HS DNA Polymerase (Stratagene, La Jolla, Calif.) two separate 50 μl reactions with each of the mutagenic primers (available upon request) was run for four cycles with the following parameters: 2.3 minutes at 92° C., 20 seconds at 58° C., 15 minutes at 68° C. and held at 4° C. After which, 25 μl of each reaction product were mixed in a single tube and then run for an additional 16 cycles. After cycling the reactions were digested with DpnI restriction enzyme (New England Biolabs, Ipswich, Mass.) for one hour and then transformed into MAX Efficiency® Stbl2™ (Invitrogen, Carlsbad, Calif.) chemically competent cells and grown at 30° C. for 9 hours. The recovered plasmids were sequenced to confirm that the mutagenesis reactions were correct. Recombinant virus was rescued as described by Estevez and co-workers (Estevez et al., 2007, supra).

Nucleotide Sequencing

All sequencing reactions were performed as previously described (Kim et al., 2006). Sequencing was done using the Applied Biosystems PRISM Fluorescent Big Dye Sequencing Kit and the ABI 3730 DNA Sequencer (ABI, Foster City, Calif.). Editing and assembly of sequence data was done using Megalign (DNASTAR, Madison, Wis.). The HN and F proteins from the recombinant Anhinga (rA), the recombinant Anhinga with the CA02 HN (rA-CAHN), the recombinant Anhinga with the CA02 HN and the LaSota fusion cleavage site (rA-CAHN-LSCL), and the recombinant Anhinga with the CA02 F and HN (rA-CAFHN) were sequenced from cDNA amplified by RT-PCR from Trizol LS (Invitrogen, Carlsbad, Calif.) extracted RNA using gene specific primers that are available upon request. Sequences have been deposited in the GenBank® under the following accession numbers: UU123456 (rA-CAHN [HN]), VV123456 (rA-CAHN [F]), WW 123456 (rA-LSCL [HN]), XX123456 (rA-LSCL [F]), YY123456 (rA-CAFHN [HN]), and ZZ123456 (rA-CAFHN [F]). Nucleotide sequences for the complete HN for Ulster (M19478), B1 (AF309418), LaSota (AY510092), TXGB (M21409) and CA02 (EF520717) are available from GenBank®. In addition the accession number for the combined sequence for the F and HN for Anhinga is AY288989. Accession numbers for the F genes of Ulster (M24694), B1 (M24695), LaSota (DQ195265), TXGB (M23407) and CA02 (EF520718) are also available from GenBank®.

Statistical Analysis

HI titers are presented as arithmetic means plus or minus standard error. VI titers are presented as arithmetic mean titers plus or minus standard error. Group means were analyzed by ANOVA with Tukey's post hoc test when indicated. Fisher exact tests performed on data indicating numbers of birds shedding. Significance is reported at the level of P≦0.05.

### Example 1

The following example illustrates that challenging vaccinated birds with a virus homologous to the vaccine reduces shedding of the challenge virus as compared to vaccinated birds challenged with a virus heterologous to the vaccine.

Chickens were separated into eight groups of six birds, wing-banded, and allowed to acclimate for two days before vaccination. Twenty-one days PV OP swabs, CL swabs and serum were collected.

Part 1. Groups were subcutaneously vaccinated with 0.5 ml of their appropriate inactivated vaccines. Group one was vaccinated with an OE made from normal allantoic fluid. Groups two, three, and four received recombinant Anhinga (rA) OE, recombinant Anhinga containing the HN gene of CA02 (rA-CAHN) OE and the homologous CA02 OE, respectively.

Part 2. Groups were vaccinated with live B1, rA, or rA-CAHN (10⁶/0.1 ml) or a sham vaccine for control birds consisting of sterile BHI.

All birds were challenged with 10^{5.7} median embryo infectious dose (EID₅₀) of CA02 virus administered in 50 μl into the right eye and 50 μl into the choana.

Results

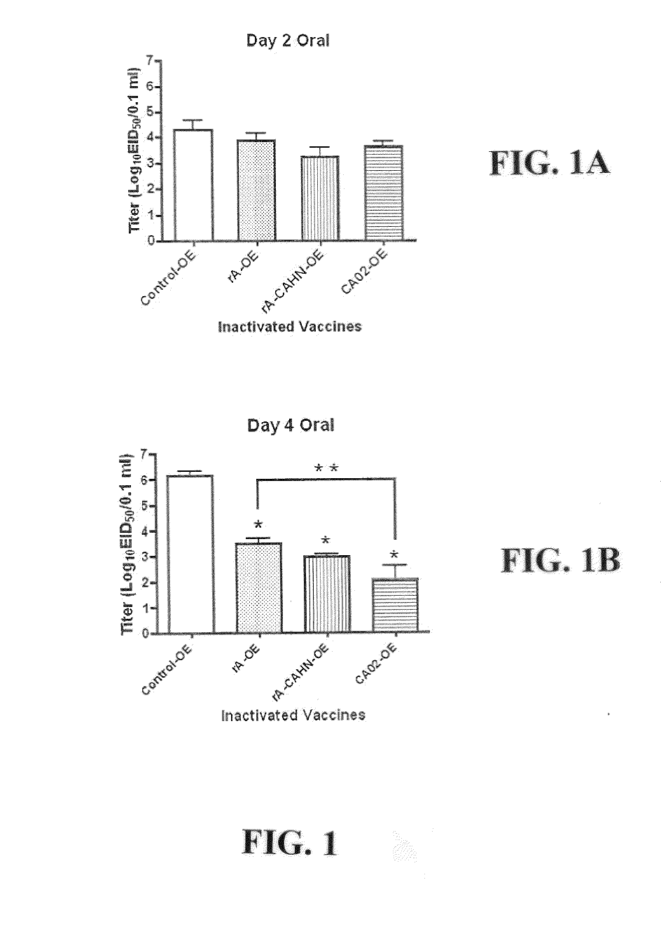

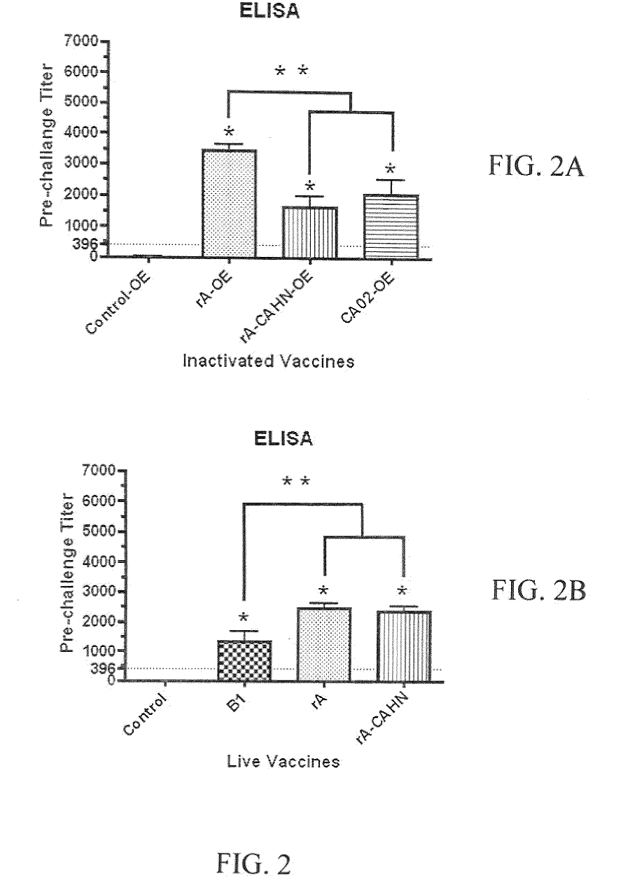

Part 1. No adverse reactions were seen in the birds vaccinated with the inactivated oil emulsion (OE) vaccines. After challenge mild conjunctivitis developed, which resolved in all of the birds except for the control sham OE vaccinated group. All birds except the control group survived challenge. All of the sham-vaccinated birds were dead by day five and upon necropsy had enlarged spleens and small hemorrhages in the thymus and mucosa of the cranial trachea. Birds vaccinated with the vaccine homologous to the challenge, CA02, shed significantly less virus in swabs of the oropharynx (OP) four days post vaccination (PV) when compared to the other inactivated vaccines (see FIG. 1) (Miller et al., 2007). Virus titers of cloacal (CL) swabs both two and four days post challenge (PC) show that all of the OE vaccine groups shed significantly less virus compared to the sham vaccinated control birds, but there was no significant difference among these vaccines (data not shown). Only rA-CAHN-OE had significantly fewer birds shedding challenge virus when compared to the controls two days PC in CL swabs (see Table 2). The pre-challenge HI and ELISA values are presented in Table 3 and FIG. 2. All vaccine groups had strongly positive ELISA and HI titers with homologous and heterologous HI antigens.

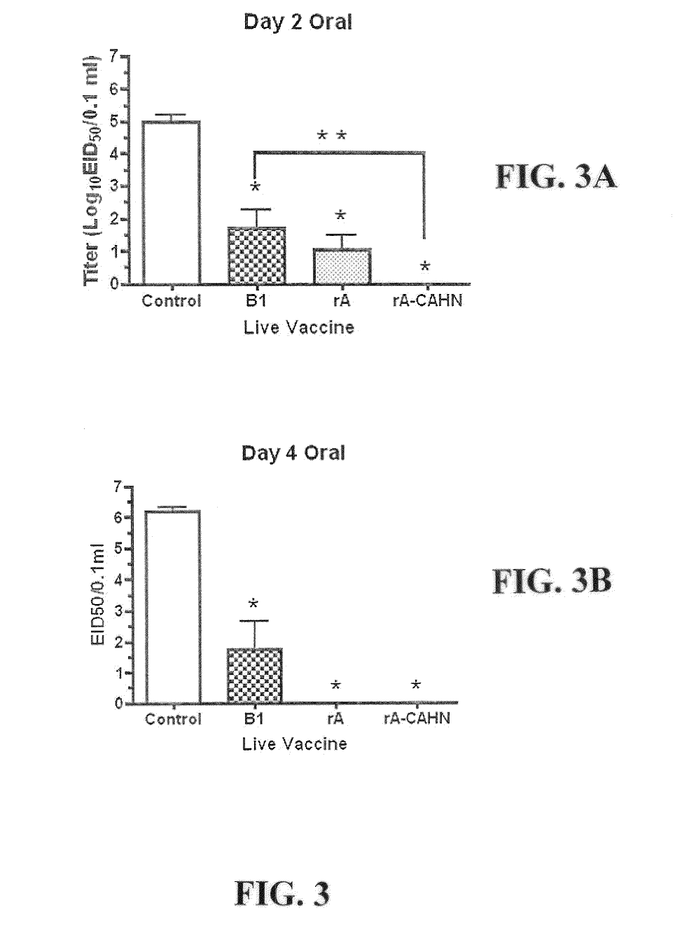

Part 2. Mild conjunctivitis in the right eye of the ND vaccinated birds from application of the live vaccine resolved after two days. Mild conjunctivitis presented again after challenge, but no other symptoms of ND were observed in the ND vaccine groups after challenge except for two birds. One bird from the B1 group died on day 4 PC and presented with hemorrhage of the cranial tracheal mucosa, a unique lesion found consistently in chickens infected with isolates from the CA02 outbreak (Wakamatsu et al., 2006). Another bird from the rA group had body tremors starting at day 2 post-challenge which resolved after day 9. The rA-CAHN group on day two PC had significantly fewer birds shedding compared to the control and B1 groups in OP swabs and on day four PC the rA and rA-CAHN groups had significantly fewer birds shedding virus in OP swabs (Table 2). All of the vaccinated birds had a significant reduction of the amount of virus shed on both two and four days PC in OP swabs when compared to the sham-vaccinated control birds (FIG. 3), but only the rA-CAHN group had significantly less virus shed than other vaccine groups day 2PC in OP swabs. CL swabs for both days PC showed results similar to the inactivated vaccines described above in experiment, part 1 (data not shown). No significant difference was seen in the protection induced in shedding of virus in OP swabs among the vaccinated groups at four days PC. However, the small number of birds per vaccine group may have obscured the differences between groups. Cross HI titers (Table 3) induced by the live vaccines in pre-challenge serum revealed B1 to have the highest antibodies to the CA02 in the cranial tracheal mucosa, a lesion typical of CA 02 infection in chickens. Two of the sham-vaccinated TXGB-challenged control birds survived challenge, but with neurological signs. Antigen and rA and rA-CAHN to have the highest to the rA antigen. Bolded values in Table 2 highlight the homologous antigens to antibodies. ELISA antibodies (FIG. 2 (b)) for live rA and rA-CAHN were similar in amounts and significantly higher than those induced by the B1 vaccine. Higher ELISA titers correlate with less virus shed in OP swabs.

TABLE 2
Experiment I - Numbers of birds shedding after challenge

| | Number of birds shedding | | | | | |
|---|---|---|---|---|---|---|
| | Days Post-Challenge | | | | | |
| | 2 | | 4 | | 9 | |
| Vaccine | Oral | Cloacal | Oral | Cloacal | Oral | Cloacal |
| Control-BHI | 6/6 | 6/6 | 6/6 | 6/6 | NS | NS |
| B1 (live) | 5/6 | 1/6* | 4/6 | 2/6 | 0/5 | 0/5 |
| rAnhinga (live) | 4/6 | 0/6* | 0/6* | 0/6* | 0/6 | 0/6 |
| rA-CAHN (live) | 0/6*# | 0/6* | 0/6* | 0/6* | 0/6 | 0/6 |
| Control-OE | 6/6 | 6/6 | 6/6 | 6/6 | NS | NS |
| rAnhinga-OE | 6/6 | 3/6 | 6/6 | 1/6* | 0/6 | 0/6 |
| rA-CAHN-OE | 6/6 | 0/6* | 6/6 | 1/6* | 0/6 | 0/6 |
| CA02-OE | 6/6 | 2/6 | 6/6 | 1/6* | 0/6 | 0/6 |

NS = No Survivors
*P < 0.05, vaccines compared to controls
#P < 0.05, rA-CAHN compared to B1

TABLE 3
Experiment I - Pre-challenge serology completed by micro-beta
HI* and ELISA**

| | HI Antigens | | | | |
|---|---|---|---|---|---|
| Vaccine | B1 | rA | rA-CAHN | CA02 | ELISA |
| B1 (live) | 123 +/− 24 | 140 +/− 41 | 106 +/− 25 | 327 +/− 106 | 1305 |
| rA (live) | 413 +/− 178 | 1227 +/− 314 | 273 +/− 85 | 653 +/− 155 | 2438 |
| rA-CHAN (live) | 533 +/− 67 | 1813 +/− 347 | 587 +/− 53 | 1067 +/− 135 | 2337 |
| rA (OE) | 267 +/− 79 | 960 +/− 320 | 127 +/− 42 | 253 +/− 84 | 3441 |
| rA-CAHN (OE) | 320 +/− 72 | 360 +/− 96 | 213 +/− 49 | 693 +/− 128 | 1632 |
| CA02 (OE) | 293 +/− 111 | 813 +/− 221 | 427 +/− 191 | 747 +/− 107 | 2045 |

*HI assays were completed with four HA units of each vaccine antigen to test pre-challenge serum of each vaccine group. Group arithmetic means with standard errors are presented and homologous responses are noted in bold.
**ELISA group arithmetic means are presented

### Example 2

Chickens were separated into five groups of six birds and five groups of nine birds, leg-banded, and allowed to acclimate for two days before vaccination. There were a total of fifteen birds for each of the four vaccine groups. Live LaSota, rA, rA-CAHN, rA-CAHN with the LaSota fusion cleavage site (rA-CAHN-LSCL) (10⁶/0.1 ml) or a sham vaccine was administered in 50 μl into the right eye and 50 μl into the choana. Four days post-vaccination (PV) OP and CL swabs were collected for VI of the vaccine virus. In addition, three birds from each of the isolators containing nine birds were randomly selected sedated and euthanized for the collection of blood, and spleens for VI. Twenty-one days PV OP and CL swabs were collected for VI and sera collected for evaluation by HI and ELISA. One of the groups for each vaccine was challenged with 10^{6.1} EID₅₀ of TXGB and the other was challenged with 10^{5.9} EID₅₀ of CA02. Challenge viruses were administered in 50 μl into the right eye and 50 μl into the choana.

### Results

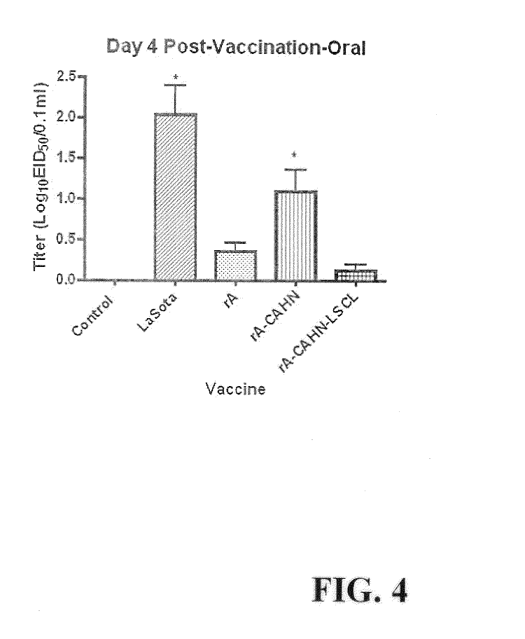

No adverse symptoms were seen after vaccination except for a mild conjunctivitis in the right eye of some of vaccinated birds that resolved by 48 hours. To ensure infections by the vaccine viruses the presence and amount of vaccine virus shed was assayed from OP and CL taken four days post vaccination (PV). Vaccine virus was recovered for all vaccine groups except from one the rA-CAHN-LSCL groups (Table 4, FIG. 4). Table 4 is pre-challenge and combines the vaccine groups. FIG. 4 lists the groups by vaccine and challenge virus. Only a few birds from the LaSota, rA, and rA-CAHN groups shed virus in CL swabs. In addition, virus was isolated and quantified from the rA and rA-CAHN vaccinated extra birds from blood, and spleen tissue at four days PV, but LaSota and rA-CAHN-LSCL were not recovered from these tissues. The median embryo infectious dose for rA and rA-CAHN isolated from the spleen at four days PV was 0.5/0.1 ml and in blood rA was also 0.5/0.1 ml EID₅₀ and rA-CAHN was 1.1/0.1 ml EID₅₀. Post challenge, mild conjunctivitis was seen in the right eye of most of the birds in both the TXGB and CA02 challenge groups. One LaSota vaccinated TXGB challenged bird died at day 5 PC after presenting with ataxia, depression and body and head tremors. No gross lesions were observed. All of the sham-vaccinated, CA02 challenged control birds died by day five PC. Upon necropsy they presented with petechial hemorrhages in the eyelid, occasional small hemorrhages in cecal tonsil and all with hemorrhage. None of the TXGB challenged sham-vaccinated control birds had gross pathological lesions, except similar to the CA02 birds, the intestinal tract was empty. All but one of the rA-CAHN-LSCL-vaccinated birds died by day 6 PC, and will be discussed at the end of this section. On day two PC OP swabs for only rA-CAHN birds challenged with CA02 show significantly fewer birds shedding virus when compared to the controls (Table 4). The CL swabs on both days two and four for LaSota, rA, and rA-CAHN challenged with both TXGB and CA02 all had significantly fewer birds shedding than the controls (Table 4). In day four PC OP swabs only, rA-CAHN birds challenged with CA02 show significantly fewer birds virus positive than the controls (Table 4).

TABLE 4
Experiment II - Number of birds shedding pre and post challenge

| | Number of birds shedding | | | | | | | | | |
|---|---|---|---|---|---|---|---|---|---|---|
| | Days post-challenge | | | | | | | | | |
| | −17^{a} | | 0 | | 2 | | 4 | | 9 | |
| Vaccine | Oral | Cloacal | Oral | Cloacal | Oral | Cloacal | Oral | Cloacal | Oral | Cloacal |
| Control (TX)^{b} | N/A | N/A | 0/6 | 0/6 | 6/6 | 6/6 | 6/6 | 6/6 | 0/4 | 1/4 |
| Control (CA) | N/A | N/A | 0/6 | 0/6 | 6/6 | 6/6 | 6/6 | 6/6 | NS | NS |
| LaSota (TX) | 4/6 | 1/6 | 0/6 | 0/6 | 4/6 | 0/6* | 4/6 | 1/6*# | 1/5^{c} | 0/5 |
| LaSota (CA) | 6/6 | 0/6 | 0/6 | 0/6 | 3/6 | 0/6* | 3/6 | 1/6*# | 0/6 | 1/6 |
| rA (TX) | 4/6 | 1/6 | 0/6 | 0/6 | 4/6 | 0/6* | 3/6 | 0/6*# | 0/6 | 0/6 |
| rA (CA) | 3/6 | 0/6 | 0/6 | 0/6 | 4/6 | 0/6* | 6/6 | 0/6*# | 0/6 | 0/6 |
| rA-CAHN-LSCL (TX) | 0/6 | 0/6 | 0/6 | 0/6 | ND | ND | 6/6 | 6/6 | NS | NS |
| rA-CAHN-LSCL (CA) | 3/6 | 0/6 | 0/6 | 0/6 | ND | ND | 6/6 | 6/6 | 1/1 | 1/1 |
| rA-CAHN (TX) | 6/6 | 2/6 | 0/6 | 0/6 | 3/6 | 0/6* | 3/6 | 0/6*# | 0/6 | 0/6 |
| rA-CAHN (CA) | 3/6 | 3/6 | 0/6 | 0/6 | 1/6* | 0/6* | 0/6*# | 0/6*# | 0/6 | 0/6 |

^{a}−17 days = 4 days post-vaccination
^{b}Vaccine (challenge virus)
^{c}one LaSota vaccinate died day 5 post challenge
*<0.05 vaccines compared to controls
#<0.05 vaccines compared to rA-CAHN-LSCL
ND: Not Done
NS: No Survivors

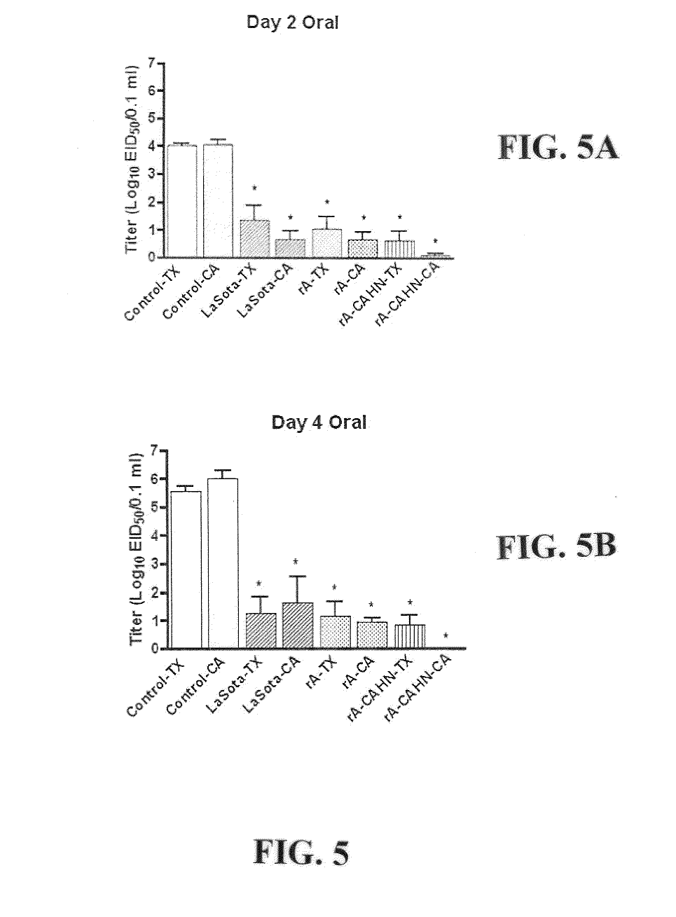

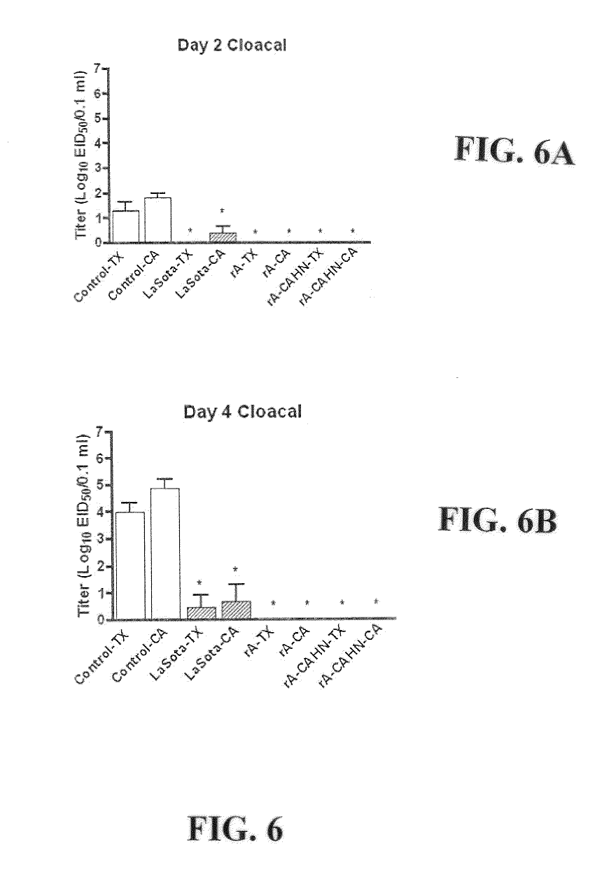

While all vaccine groups, except rA-CAHN-LSCL, shed less virus than the controls, a trend was seen for the homologous vaccine/challenge virus combinations to shed less virus compared to the same vaccine with the genotypically heterologous challenge virus. No statistical significance was noted comparing vaccines to each other (FIGS. 5 and 6). LaSota vaccinated birds had the highest HI antibodies to their homologous antigen (Table 5). The rA and rA groups had HI antibodies highest to CA02 antigen. The rA-CAHN-LSCL group while positive (greater than 16) only had HI levels of 22 to the LaSota antigen and 24 to the CA02 antigen (Table 5).

TABLE 5
Experiment II - Pre-challenge serology completed by micro-beta
HI* and ELISA**

| | HI Antigens | | | | | |
|---|---|---|---|---|---|---|
| Vaccine | LaSota | rA | rA-CAHN | TX | CA02 | ELISA |
| LaSota | 124 +/− 24 | 81 +/− 19 | 129 +/− 27 | 108 +/− 22 | 84 +/− 13 | 1279 +/− 1195 |
| rA | 421 +/− 73 | 875 +/− 135 | 560 +/− 109 | 355 +/− 100 | 896 +/− 215 | 3605 +/− 1941 |
| rA-CAHN | 1045 +/− 156 | 1109 +/− 85 | 1365 +/− 182 | 877 +/− 135 | 3499 +/− 320 | 4252 +/− 1313 |
| rA-CAHN-LSCL | 22 +/− 11 | ND | ND | ND | 24 +/− 7 | 370 +/− 120 |

*HI assays were completed with four HA units of each vaccine antigen to test pre-challenge serum of each vaccine group. Group arithmetic means with standard errors are presented and homologous responses are noted in bold.
**ELISA group arithmetic means are presented
ND = Not Done

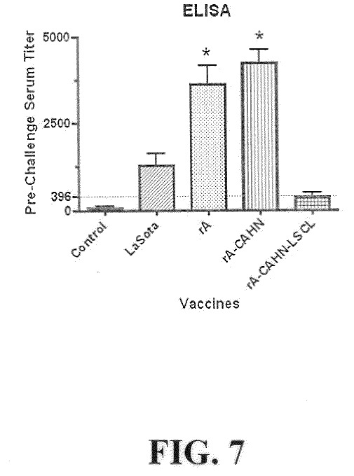

Highest to lowest the ELISA values for the vaccine groups were rA-CAHN, rA, LaSota and rA-CAHN-LSCL. The rA-CAHN-LSCL group was negative on ELISA with a mean antibody value of 370, less than the 396 cut off value suggested by the manufacturer (FIG. 7). Not noted on FIG. 7 was significance seen between 1) LaSota, rA, and rA-CAHN vaccinated birds and rA-CAHN-LSCL and 2) LaSota vaccine group and rA. Interestingly, individual rA-CAHN-LSCL-vaccinated birds that did have ELISA antibodies between 400 and 1495 died after challenge and the one surviving bird had an ELISA titer of 311 (data not shown). The LaSota-vaccinated, TXGB-challenged bird that died had an ELISA titer of 166, however there were other birds in the group with negative ELISA titers that lived and showed no symptoms of ND. The back-titer of the rA-CAHN-LSCL had an EID₅₀ of 10^{5.1}/bird, almost a log lower than the target dose of an EID₅₀ of 10^{6.0}/bird. All of the rA-CAHN-LSCL birds challenged with TXGB died by day six post-challenge and five of the six CA02 challenged birds died by day six.

### Example 3

Chickens were separated into five groups of ten birds and five groups of eleven birds, leg-banded, and allowed to acclimate for two days before vaccination. There were a total of twenty-one birds for each of the five vaccine groups. Live Ulster, LaSota, rA, rA-CAHN and rA-CAFHN (10⁶/0.1 ml) or a sham vaccine was administered in 50 μl into the right eye and 50 μl into the choana. An additional group of 11 birds was vaccinated with rA-CAHN-LSCL. Because of the poor response of this vaccine from experiment II, these birds were challenged only with CA02. Four days PV OP and CL swabs were collected for VI of the vaccine virus from all birds. In addition one bird in each of the isolators containing eleven birds was randomly selected for euthanasia for the collection of lungs, and tracheas for each of the six groups for VI. Twenty-one days PV OP swabs, CL swabs and serum were collected. One of the groups for each vaccine was challenged with 10^{6.3} EID₅₀ of TXGB and the other was challenged with 10^{7.1} EID₅₀ of CA02. Both were administered in 50 μl into the right eye and 50 μl into the choana.

### Results

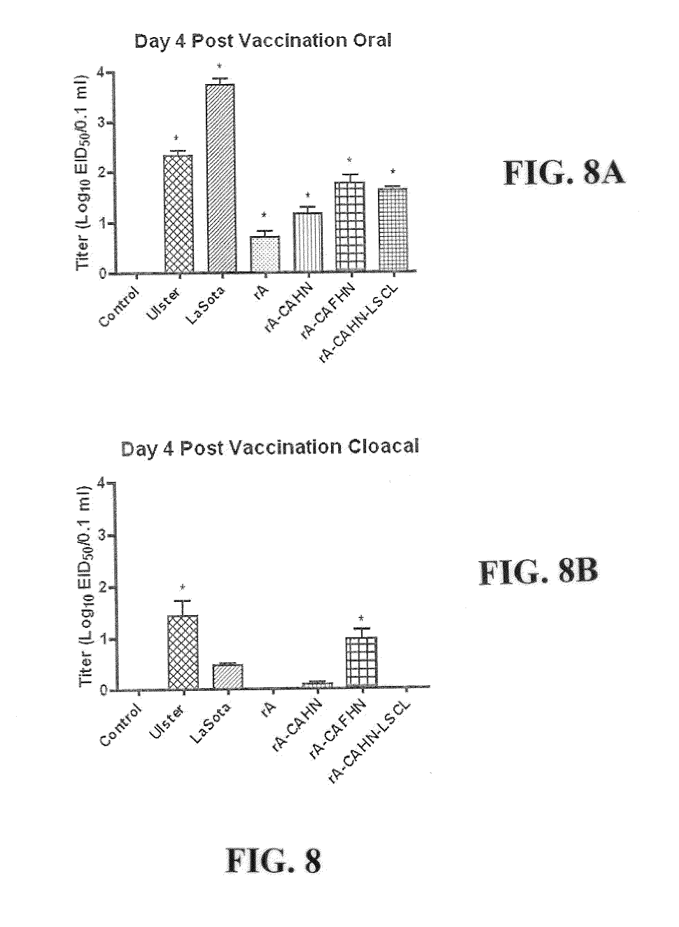

The live vaccines caused mild conjunctivitis that resolved by day two PV as seen in prior experiments. All vaccine viruses were recovered from OP swabs collected at four days PV, but only Ulster, LaSota and rA-CAHN were recovered from CL swabs (FIG. 8, Table 6). Additionally, tracheas and lungs were harvested for virus isolation to identify virus distribution to these organs post-vaccination (Table 6). Post-challenge mild conjunctivitis resolved by day two in the ND vaccinates. Two days PC the sham vaccinated CA02 challenged controls had moderate conjunctivitis that worsened over time and the sham-vaccinated TXGB challenged controls had mild conjunctivitis that resolved by the next day. The sham vaccinated CA02 challenged controls were dead by six days PC and upon necropsy had enlarged spleens, bilateral petechia of the conjunctiva, mild hemorrhage in some thymic tissues and cranial hemorrhage of the trachea. Sham vaccinated TXGB challenged controls died by day eleven PC and upon necropsy had no hemorrhage or other lesions. All ND-vaccinated birds survived challenge without showing symptoms of ND except for two rA-CAHN-LSCL vaccinated birds. These two rA-CAHN-LSC-vaccinated birds had no symptoms of ND until four days PC when bilateral conjunctivitis, diarrhea, and depression developed. At seven days PC one of these birds looked clinically normal, and the other had developed a head tilt that progressed to severe torticollis by eleven days PC. Upon euthanasia and necropsy this bird had bilateral petechia in conjunctival tissues but no other gross lesions.

Residual vaccine virus was isolated sporadically from all of the vaccine groups on the day of challenge (Table 7). The day two PC OP swabs showed LaSota-vaccinated birds challenged with TXGB, rA-CAHN-vaccinated birds challenged with CA02, and rA-CAFHN-vaccinated birds challenged with either TXGB or CA02 with significantly fewer birds shedding virus compared with the sham-vaccinated groups (Table 7).

TABLE 7
Experiment III - Number of birds shedding virus pre and post
challenge

| | Number of birds shedding | | | | | | | | | |
|---|---|---|---|---|---|---|---|---|---|---|
| | Days post-challenge | | | | | | | | | |
| | −17^{a} | | 0 | | 2 | | 4 | | 9 | |
| Vaccine | Oral | Cloacal | Oral | Cloacal | Oral | Cloacal | Oral | Cloacal | Oral | Cloacal |
| Control (TX)^{b} | 0/10 | 0/10 | 0/10 | 0/10 | 10/10 | 10/10 | 10/10 | 10/10 | 0/2^{c} | 1/2 |
| Control (CA) | 0/10 | 0/10 | 0/10 | 0/10 | 10/10 | 10/10 | 10/10 | 10/10 | NS | NS |
| Ulster (TX) | 10/10 | 8/10 | 0/10 | 1/10 | 10/10 | 0/10* | 6/10 | 1/10* | 0/10 | 1/10 |
| Ulster (CA) | 10/10 | 7/10 | 1/10 | 2/10 | 10/10 | 1/10* | 9/10 | 6/10* | 0/10 | 0/10 |
| LaSota (TX) | 10/10 | 9/10 | 0/10 | 2/10 | 5/10*^{#} | 1/10* | 1/10* | 0/10* | 2/10 | 0/10 |
| LaSota (CA) | 10/10 | 9/10 | 0/10 | 1/10 | 10/10 | 1/10* | 2/10* | 1/10* | 0/10 | 0/10 |
| rA (TX) | 10/10 | 0/10 | 0/10 | 1/10 | 7/10 | 1/10* | 6/10 | 3/10* | 2/10 | 0/10 |
| rA (CA) | 10/10 | 0/10 | 1/10 | 0/10 | 7/10 | 2/10* | 3/10* | 0/10* | 0/10 | 0/10 |
| rA-CAHN (TX) | 10/10 | 1/10 | 1/10 | 0/10 | 6/10 | 2/10* | 2/10* | 0/10* | 0/10 | 1/10 |
| rA-CAHN (CA) | 10/10 | 2/10 | 0/10 | 0/10 | 3/10*^{#} | 0/10* | 1/10* | 2/10* | 0/10 | 0/10 |
| rA-CAFHN (TX) | 10/10 | 5/10 | 2/10 | 1/10 | 5/10*^{#} | 0/10* | 3/10* | 0/10* | 0/10 | 0/10 |
| rA-CAFHN (CA) | 10/10 | 5/10 | 1/10 | 0/10 | 4/10*^{#} | 0/10* | 4/10* | 0/10* | 0/10 | 0/10 |
| rA-CAHN-LSCL (CA) | 10/10 | 0/10 | 0/10 | 3/10 | 10/10 | 9/10 | 10/10 | 10/10 | 1/10 | 2/10 |

^{a}17 days before challenge = 4 days after vaccination
^{b}Vaccine (Challenge virus)
^{c}all Control (TX) birds expired by day 10
NS: No Survivors
*<0.05 vaccines compared to controls and rA-CAHN-LSCL (CA)
^{#}<0.05 from LaSota (CA)

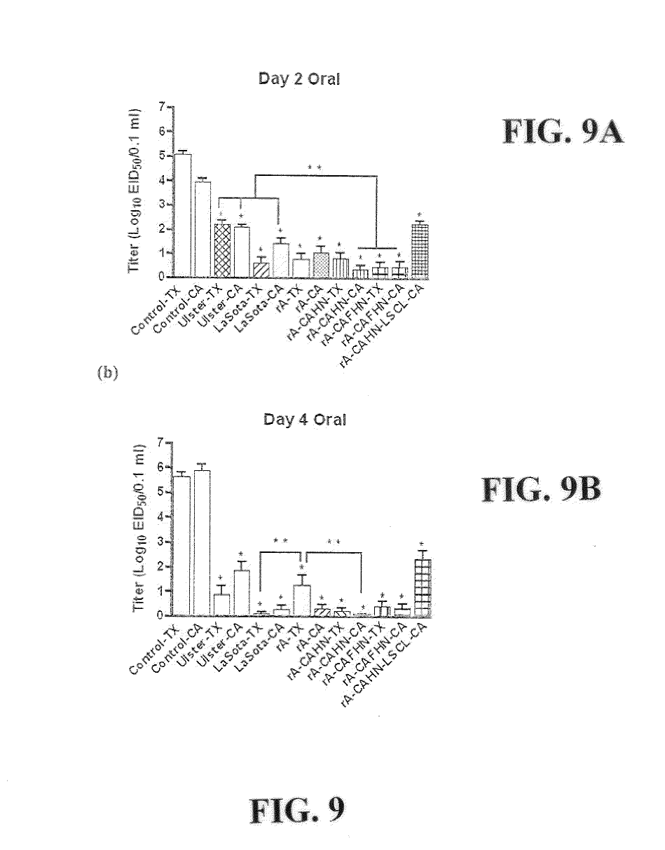

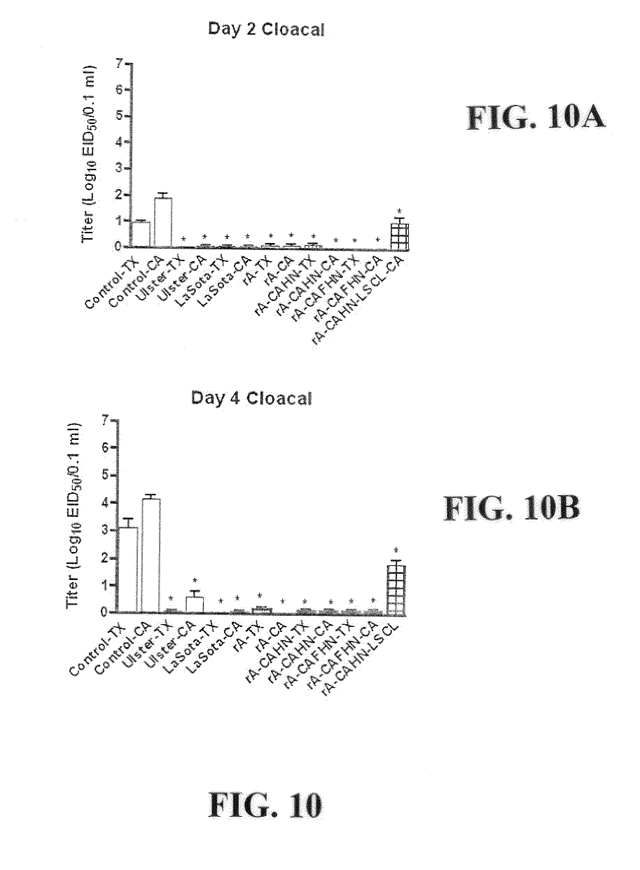

Day two and four PC CL swabs show that all vaccine groups except rA-CAHN-LSCL had significantly fewer birds shedding compared to the controls. Day four OP swabs show LaSota, rA-CAHN, and rA-CAFHN vaccinated birds challenged with TXGB and LaSota, rA, rA-CAHN, and rA-CAFHN vaccinated birds challenged with CA02 had significantly fewer birds shedding per group compared to the controls (Table 7). FIG. 9 (a) shows that all vaccine groups on average shed significantly less virus than the controls on day two PC in OP swabs. In addition, rA-CAHN vaccinated birds challenged with CA02 and rA-CAFHN vaccinated birds challenged with either TXGB or CA02 shed significantly less virus compared to both Ulster vaccine groups and to LaSota vaccinated birds challenged with CA02. Other significance not noted on FIG. 9 (a) are 1) LaSota, rA, and rA-CAHN vaccinated birds challenged with TXGB and rA vaccinated birds challenged with CA02 shed significantly less than both Ulster groups, and 2) all vaccine groups except the Ulster groups shed significantly more virus than the rA-CAHN-LSCL vaccine group. Even genotypically heterologous vaccine/challenge combinations provided better shed from virulent challenge compared with the Ulster-vaccinates. Four day PC OP swabs (FIG. 9 (b)) have similar results in that all vaccine groups shed less virus than the sham vaccinated control groups. LaSota vaccinated birds challenged with TXGB and rA-CAHN vaccinated birds challenged with CA02 shed significantly less than rA-vaccinated birds challenged with TXGB. Other significance not noted on FIG. 9 (b) is that all vaccine groups except Ulster shed significantly less than the rA-CAHN-LSCL group. FIG. 10 shows that all vaccines in CL swabs on days two and four PC shed significantly less than the controls except for the rA-CAHN-LSCL group which did not shed significantly less than sham-vaccinated controls challenged with TXGB. All vaccinated birds shed significantly less virus compared to rA-CAHN-LSCL, which is not noted in FIGS. 4.10 (a) and (b).

The three recombinants, rA, rA-CAHN and rA-CAFHN, induced strong HI antibody responses to the CA02 and the TXGB antigens (Table 8).

TABLE 8
Experiment III - Pre-challenge serology completed by micro-beta
HI* and ELISA**

| | HI Antigens | | | | |
|---|---|---|---|---|---|
| Vaccine | Ulster | LaSota | rA | rA-CAHN | rA-CAFHN |
| Ulster | 94 +/− 13 | 54 +/− 9 | 66 +/− 9 | 8 +/− 14 | 195 +/− 34 |
| LaSota | 115 +/− 13 | 162 +/− 17 | 101 +/− 14 | 131 +/− 20 | 306 +/− 184 |
| rA | 922 +/− 145 | 726 +/− 146 | 912 +/− 128 | 832 +/− 114 | 1485 +/− 133 |
| rA- | 1766 +/− 114 | 1920 +/− 90 | 1690 +/− 112 | 1690 +/− 112 | 1946 +/− 70 |
| CAHN | | | | | |
| rA- | 1638 +/− 132 | 1203 +/− 119 | 1075 +/− 104 | 1203 +/− 119 | 2048 +/− 0 |
| CAFHN | | | | | |
| rA- | 21 +/− 6 | 19 +/− 8 | 24 +/− 5 | 12 +/− 3 | 70 +/− 10 |
| CAHN-LSCL | | | | | |
| | | HI Antigens | | | |
| | | rA-CAHN- | | | |
| | Vaccine | LSCI | TX | CA02 | ELISA |
| | Ulster | 57 +/− 35 | 131 +/− 33 | 101 +/− 14 | 1218 +/− 14 |
| | LaSota | 68 +/− 3 | 141 +/− 31 | 128 +/− 27 | 3763 +/− 464 |
| | rA | 1024 +/− 129 | 1082 +/− 175 | 1510 +/− 141 | 5652 +/− 627 |
| | rA- | 1280 +/− 136 | 1920 +/− 90 | 1894 +/− 84 | 7705 +/− 582 |
| | CAHN | | | | |
| | rA- | 1331 +/− 141 | 1305 +/− 146 | 1946 +/− 70 | 6492 +/− 415 |
| | CAFHN | | | | |
| | rA- | 21 +/− 3 | 46 +/− 10 | 38 +/− 11 | 288 +/− 622 |
| | CAHN-LSCL | | | | |

*HI assays were completed with four HA units of each vaccine antigen to test pre-challenge serum of each vaccine group. Group arithmetic means with standard errors are presented and homologous responses are noted in bold.
**ELISA group arithmetic means are presented

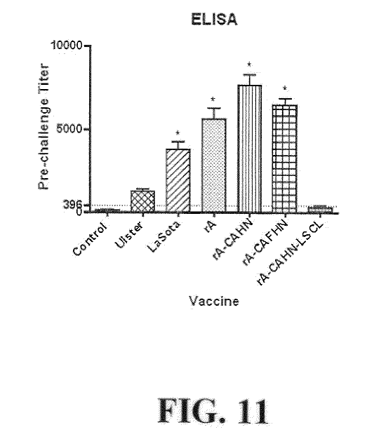

The rA-CAHN-LSCL group had the highest antibodies to rA-CAFHN with a mean of 70 and a mean HI titer of 38 to the CA02 antigen. All vaccines had robust ELISA titers (FIG. 11) except for rA-CAHN-LSCL, which had a mean value of 288, below the cut of for a positive response. Even without a positive ELISA and with low HI antibody titers all of the rA-CAHN-LSCL-vaccinate birds survived challenge. Other significance not noted on FIG. 11: 1) LaSota is significantly lower than rA, rA-CAHN, rA-CAFHN, and significantly higher than rA-CAHN-LSCL, 2) rA is significantly different lower than rA-CAHN, rA-CAFHN, and significantly higher than rA-CAHN-LSCL, 3) Ulster is significantly lower than LaSota, rA, rA-CAHN, and rA-CAFHN, and 4) rA-CAHN-LSCL is significantly lower than rA-CAHN and rA-CAFHN.

### Example 4

Four vaccine groups each with twenty birds and two control groups with six birds each were housed in the 12 isolators. Size limitations of the isolators forced uneven cage numbers for the different groups. That is; groups 1 (LaSota vaccine/TXGB challenge) and 4 (rA-CAFHN vaccine/TXGB challenge) consisted of two isolators each with ten birds and groups 2 (LaSota vaccine/CA02 challenge) and 3 (rA-CAFHN/CA02 challenge) consisted of one isolator with ten birds and two isolators with five birds each. Two more isolators housed groups 6 and 7, each with six birds vaccinated with control vaccines. A sham vaccine, live LaSota, or rA-CAFHN at a dose of 10⁶/0.1 ml was administered in 50 μl into the right eye and 50 μl into the choana. The control groups received 100 μl of BHI administered in the same manner. Twenty-one days PV OP swabs, CL swabs and serum were collected. One of the groups for each vaccine was challenged with 106.5 EID₅₀ of TXGB and the other was challenged with 10^{6.9} EID₅₀ of CA02. Both were administered in 50 μl into the right eye and 50 μl into the choana.

### Results

Mild conjunctivitis resolved within two days PV in ND vaccinated birds. After challenge mild conjunctivitis in ND vaccinated birds resolved within one day. Sham-vaccinated controls challenged with TXGB also had mild conjunctivitis that resolved after one day and the group was dead by ten days PC after showing neurological signs, but with no lesions observed upon necropsy. Sham-vaccinated controls challenged with CA02 developed conjunctivitis and depression with severe respiratory symptoms leading to death by day five PC. Upon necropsy, hemorrhage in the conjunctiva and the cranial trachea mucosa was observed. Day two PC OP swabs show both LaSota vaccinated TXGB challenged and rA-CAFHN vaccinated CA02 challenged groups had significantly fewer birds shedding compared to controls (Table 9).

TABLE 9
Experiment IV - Number of birds shedding post-challenge

| | Number of birds shedding | | | | | | | |
|---|---|---|---|---|---|---|---|---|
| | Days post-challenge | | | | | | | |
| | 0 | | 2 | | 4 | | 9 | |
| Vaccine | Oral | Cloacal | Oral | Cloacal | Oral | Cloacal | Oral | Cloacal |
| Control (TX)^{a} | 0/6 | 0/6 | 6/6 | 6/6 | 6/6 | 6/6 | 0/1 | NS |
| Control (CA) | 0/6 | 0/6 | 6/6 | 6/6 | 6/6 | 6/6 | NS | NS |
| LaSota (TX) | 1/20 | 1/20* | 5/20*⊕ | 2/20* | 2/20* | 0/20*⊕ | 1/20 | 0/20 |
| LaSota (CA) | 0/20 | 7/20* | 14/20 | 0/20* | 5/20* | 5/20* | 0/20 | 1/20 |
| rA-CAFHN (TX) | 0/20 | 0/20* | 14/20 | 0/20* | 7/20* | 3/20* | 0/20 | 2/20 |
| rA-CAFHN (CA) | 0/20 | 0/20* | 4/20*# | 3/20* | 0/20*# | 1/20* | 0/20 | 1/20 |

^{a}Vaccine (Challenge)
*P ≦ 0.05 significance from control vaccines
⊕P ≦ 0.05 significance from LaSota (CA)
#P ≦ 0.05 significance from rA-CAFHN (TX)

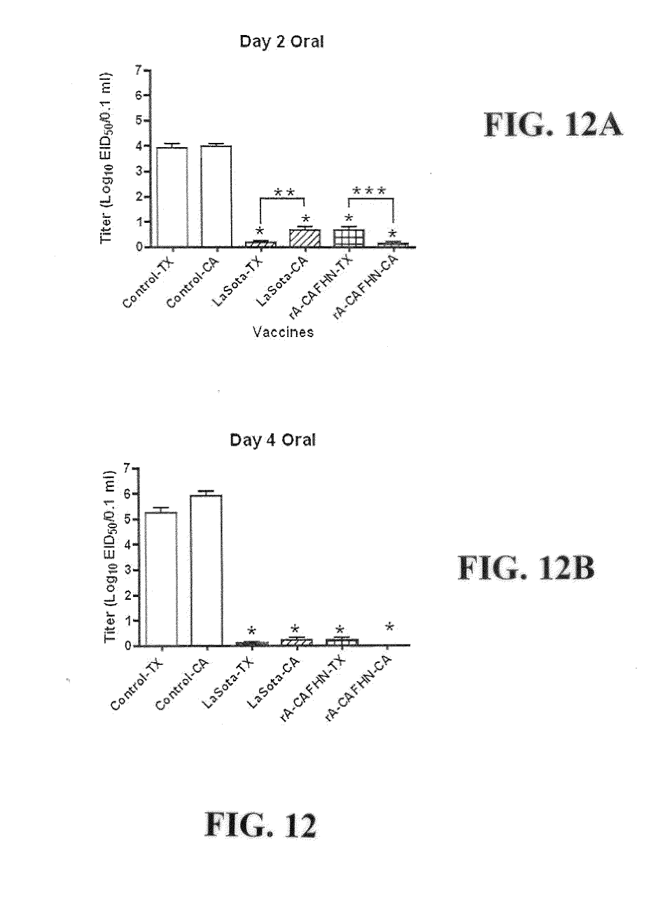

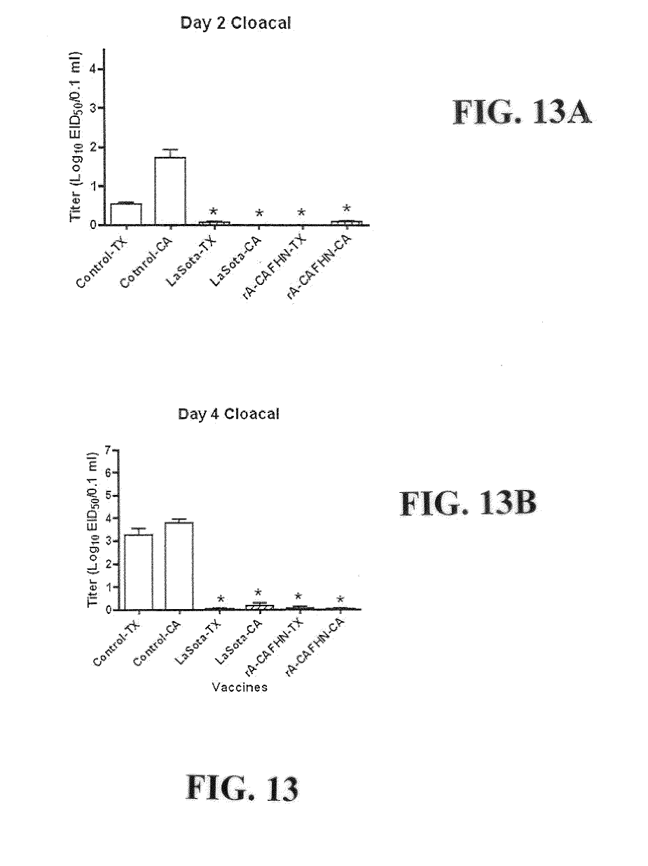

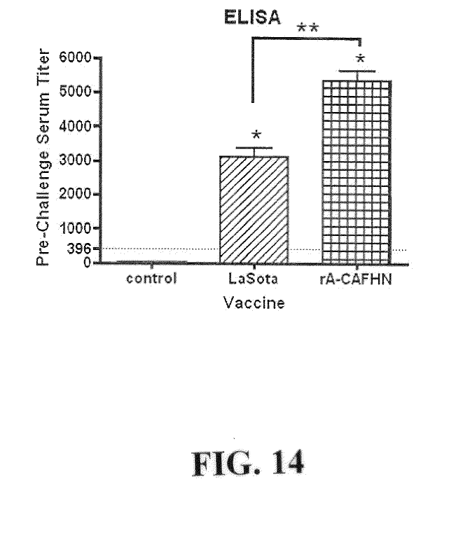

More notable rA-CAFHN vaccinated birds challenged with CA02 have significantly fewer birds shedding than the rA-CAFHN vaccinated TXGB-challenged birds. In two day PC CL swabs both LaSota vaccinated birds challenged with TXGB and rA-CAFHN-vaccinated birds challenged with CA02 have significantly fewer birds shedding than controls. Four day PC OP swabs show LaSota-vaccinated TXGB-challenged birds and rA-CAFHN-vaccinated CA02-challenged birds had significantly fewer birds shedding compared to controls (Table 9). The rA-CAFHN-vaccinated CA02-challenged had significantly fewer birds shedding when compared to rA-CAFHN-vaccinated TXGB-challenged birds. In four-day PC CL swabs all vaccine groups had significantly fewer birds shedding when compared to the control groups and LaSota-vaccinated TXGB-challenged birds had significantly fewer birds shedding when compared to the LaSota-vaccinated CA02-challenged group. FIG. 12 (a) shows that all vaccine groups shed on average less virus when compared to sham-vaccinated controls. More importantly the LaSota vaccinated birds challenged with TXGB shed significantly less virus compared to the LaSota-vaccinated birds challenged with CA02 and the rA-CAFHN-vaccinated birds challenged with CA02 shed significantly less virus compared to the rA-CAFHN-vaccinated birds challenged with TXGB. At four days PC OP swabs show all vaccine groups shed significantly less virus than the control groups (FIG. 12 (b)). For days two and four PC in CL swabs significance was seen between all vaccine groups compared to the controls, but no significance was seen between the vaccine groups (FIG. 13). The mean HI antibody titer of the rA-CAFHN vaccine group was six-fold higher to the CA02 antigen and four-fold higher to TXGB compared to the mean antibody titers induced by LaSota (Table 10). ELISA titers for both vaccine groups were robust with both significantly higher than the control groups, however the rA-CAFHN vaccine group had a mean ELISA titer significantly higher than the LaSota ELISA titer (FIG. 14).

It is understood that the examples and embodiments described herein are for illustrative purposes only and that various modifications or changes in light thereof will be suggested to persons skilled in the art and are to be included within the spirit and purview of this application and scope of the appended claims.

What is claimed is:

1. A Newcastle Disease vaccine, the vaccine comprising: a recombinant attenuated virulence Newcastle disease virus;
wherein
the recombinant attenuated virulence Newcastle disease virus comprises at least one substituted gene that is homologus to a corresponding gene of a virulent challenge strain of Newcastle disease virus, and
wherein
the at least one substituted gene is a member selected from the group consisting of a hemagglutinin neuraminidase (HN) gene and a fusion (F) gene or a combination thereof.

2. The Newcastle Disease vaccine of claim 1, wherein the Newcastle Disease vaccine is a live vaccine.

3. The Newcastle Disease vaccine of claim 2, wherein the recombinant attenuated virulence Newcastle disease virus is a member selected from the group consisting of lentogenic Newcastle disease viruses and mesogenic Newcastle disease viruses.

4. The Newcastle Disease vaccine of claim 3, wherein the recombinant attenuated virulence Newcastle disease virus is a lentogenic Newcastle disease virus that is a member selected from the group consisting of: Ulster/196, B1/1947, and LaSota/1946.

5. The Newcastle Disease vaccine of claim 3, wherein the recombinant low virulence Newcastle disease virus is a mesogenic Newcastle disease virus.

6. The live Newcastle Disease vaccine of claim 5, wherein the mesogenic Newcastle disease virus is recombinant Anhinga strain.

7. The Newcastle Disease vaccine of claim 6, wherein the recombinant Anhinga strain comprises a substituted HN gene.

8. The Newcastle Disease vaccine of claim 7, wherein the recombinant Anhinga strain further comprises a substituted F gene.

9. A method for stimulating immunity to Newcastle disease in poultry, the method comprising:
vaccinating poultry with a vaccine comprising a recombinant attenuated virulence Newcastle disease virus;
wherein
the recombinant attenuated virulence Newcastle disease virus comprises at least one substituted gene homologus to a virulent challenge strain of Newcastle disease virus, and
wherein
the at least one substituted gene homologus to the virulent challenge strain of Newcastle disease virus is a member selected from the group consisting of a hemagglutinin neuraminidase (HN) gene and a fusion (F) gene or a combination thereof.

10. The method of claim 9, wherein the vaccine is a live vaccine.

11. The method of claim 10, wherein the vaccinating is achieved through mass vaccination.

12. The method of claim 11, wherein mass vaccination is achieved by providing the vaccine in drinking water.

13. The method of claim 11, wherein mass vaccination is achieved by contacting the poultry with an aerosol spray comprising the vaccine.

14. The method of claim 9, wherein the vaccinating is achieved through subcutaneous injection.

15. The method of claim 14, wherein the vaccine is inactivated.

16. The method of claim 14, wherein the vaccine is live.
